(19) United States
(12) Patent Application Publication
(10) Pub. No.: US 2026/0345059 A1
(43) Pub. Date: Nov. 5, 2026

(54) MULTIFUNCTIONAL BODY-SCALE DEVICE PLATFORM

(71) Applicant: 4SIGHT2020 INC., Middletown, DE (US)

(72) Inventors: Arun SHARMA, Montville, NJ (US); Sudipta GHOSH, Kolkata (IN); Mudit SHARMA, Delhi (IN); Abhilash G DUTT, Nagar (IN)

(73) Assignee: 4SIGHT2020 INC., Middletown, DE (US)

(21) Appl. No.: 19/487,304

(22) PCT Filed: May 24, 2024
(86) PCT No.: PCT/IB2024/055094

§ 371 Date: Nov. 24, 2025

Related U.S. Application Data
(60) Provisional application No. 63/504,327, filed on May 25, 2023.

(51) Int. Cl. A61B 5/375; A61B 5/00; A61B 5/053; A61B 5/11; A61B 5/1172; A61B 5/1455; A61B 5/259

(52) U.S. Cl. CPC A61B 5/375; A61B 5/053; A61B 5/1112; A61B 5/1172; A61B 5/1455; A61B 5/259; A61B 5/6887; A61B 5/7203

(57) ABSTRACT

A method and a system for determining user-specific risk assessment scores using a multifunctional body device. The method includes collecting sensor data from multifunctional body device (MBD), wherein the sensor data includes impedance cardiography (ICG) sensor data, electrocardiogramaensor data, and bioimpedance sensor data; preprocessing the ICG sensor data and the ECG sensor data to remove artifacts, by applying a first machine learning algorithm, wherein the artifacts have signal interferences between the ICG sensor data and the ECG sensor data; determining a plurality of health parameters by applying at least one evaluation model to the preprocessed ICG sensor data, preprocessed ECG sensor data, and theWe bioimpedance data; generating at least one risk score for a specific user based on the determined plurality of health parameters and user profile data, wherein the user profile data are unique to a respective user and includes personal data and historical data.

### CROSS-REFERENCE TO RELATED APPLICATIONS

This application claims the benefit of U.S. Provisional Application No. 63/504,327 filed on May 25, 2023, the contents of which are hereby incorporated by reference.

### TECHNICAL FIELD

The present disclosure generally relates to body-scale device platforms and more particularly to a multifunctional body-scale device that determines combination of physiological conditions.

### BACKGROUND

Mobile devices have demonstrated advantages of combining separate hand-held electronic devices to form multifunctional devices. By having a single multifunctional device, a user is not burdened with obtaining, purchasing, maintaining, and working with multiple devices. Similar approaches can be applied to body-scale devices to create a single multifunctional body-scale device that can be easily embedded within an individual's environment thereby allowing increased usage of the device, greater frequency of data collection, increased data types for data collection, and access to a holistic view of an individual's body-related measurements.

However, it has been identified that for each new device functionality that is added to a single device, the complexity and size of the device is bound to increase. In some cases, combining devices may result in redundant hardware components, which allow components to be used for multiple, different device functionalities. In other cases, certain hardware components are distinct to each device and therefore additional space and connectivity must be made available. In addition, separate detectors (e.g., sensors) may be configured for each device functionality, which can further increase size of the device.

Furthermore, each device functionality typically has its own programming or application software and, therefore, the multifunctional device must be designed with enough memory to accommodate all the various software components. Therefore, a notable trade-off exists between keeping the footprint small and complexity low while still maximizing the functionality of the device.

It would therefore be advantageous to provide a solution that would overcome the challenges noted above.

### SUMMARY

A summary of several example embodiments of the disclosure follows. This summary is provided for the convenience of the reader to provide a basic understanding of such embodiments and does not wholly define the breadth of the disclosure. This summary is not an extensive overview of all contemplated embodiments, and is intended to neither identify key or critical elements of all embodiments nor to delineate the scope of any or all aspects. Its sole purpose is to present some concepts of one or more embodiments in a simplified form as a prelude to the more detailed description that is presented later. For convenience, the term “some embodiments” or “certain embodiments” may be used herein to refer to a single embodiment or multiple embodiments of the disclosure.

Certain embodiments disclosed herein include a method for simultaneously determining a plurality of health parameters. The method comprises: collecting sensor data from a multifunctional body device (MBD), wherein the sensor data include impedance cardiography (ICG) sensor data and electrocardiogramaensor data; preprocessing the ICG sensor data and the ECG sensor data to remove artifacts, by applying a first machine learning algorithm, from each of the ICG sensor data and the ECG sensor data, wherein the artifacts have signal interferences between the ICG sensor data and the ECG sensor data; extracting sensor features from each of the preprocessed ICG sensor data and the ECG sensor data, wherein the sensor features identify sensor data characteristics for each of the ICG sensor data and the ECG sensor data; determining the plurality of health parameters by applying an evaluation model to the extracted sensor features from the ICG sensor data and the ECG sensor data, wherein the evaluation model is a multi-layer neural network model; aggregating the determined plurality of health parameters to determine a trend of the plurality of health parameters, wherein the trend is monitored over time; and generating a report on the determined trend of the plurality of health parameters, wherein the generated report is caused to be displayed to a user.

Certain embodiments disclosed herein also include a non-transitory computer readable medium having stored thereon causing a processing circuitry to execute a process, the process comprising: collecting sensor data from a multifunctional body device (MBD), wherein the sensor data include impedance cardiography (ICG) sensor data and electrocardiogramaensor data; preprocessing the ICG sensor data and the ECG sensor data to remove artifacts, by applying a first machine learning algorithm, from each of the ICG sensor data and the ECG sensor data, wherein the artifacts have signal interferences between the ICG sensor data and the ECG sensor data; extracting sensor features from each of the preprocessed ICG sensor data and the ECG sensor data, wherein the sensor features identify sensor data characteristics for each of the ICG sensor data and the ECG sensor data; determining the plurality of health parameters by applying an evaluation model to the extracted sensor features from the ICG sensor data and the ECG sensor data, wherein the evaluation model is a multi-layer neural network model; aggregating the determined plurality of health parameters to determine a trend of the plurality of health parameters, wherein the trend is monitored over time; and generating a report on the determined trend of the plurality of health parameters, wherein the generated report is caused to be displayed to a user.

Certain embodiments disclosed herein also include a system for simultaneously determining a plurality of health parameters. The system comprises: a processing circuitry; and a memory, the memory containing instructions that, when executed by the processing circuitry, configure the system to: collect sensor data from a multifunctional body device (MBD), wherein the sensor data include impedance cardiography (ICG) sensor data and electrocardiogramaensor data; preprocess the ICG sensor data and the ECG sensor data to remove artifacts, by applying a first machine learning algorithm, from each of the ICG sensor data and the ECG sensor data, wherein the artifacts have signal interferences between the ICG sensor data and the ECG sensor data; extract sensor features from each of the preprocessed ICG sensor data and the ECG sensor data, wherein the sensor features identify sensor data characteristics for each of the ICG sensor data and the ECG sensor data; determine the plurality of health parameters by applying an evaluation model to the extracted sensor features from the ICG sensor data and the ECG sensor data, wherein the evaluation model is a multi-layer neural network model; aggregate the determined plurality of health parameters to determine a trend of the plurality of health parameters, wherein the trend is monitored over time; and generate a report on the determined trend of the plurality of health parameters, wherein the generated report is caused to be displayed to a user.

Certain embodiments disclosed herein include a method for determining user-specific risk assessment scores. The method comprises: collecting sensor data from a multifunctional body device (MBD), wherein the sensor data includes impedance cardiography (ICG) sensor data, electrocardiogramaensor data, and bioimpedance sensor data; preprocessing the ICG sensor data and the ECG sensor data to remove artifacts, by applying a first machine learning algorithm, from each of the ICG sensor data and the ECG sensor data, wherein the artifacts have signal interferences between the ICG sensor data and the ECG sensor data; determining a plurality of health parameters by applying at least one evaluation model to the preprocessed ICG sensor data, preprocessed ECG sensor data, and the bioimpedance data; generating at least one risk score for a specific user based on the determined plurality of health parameters and user profile data, wherein the user profile data are unique to a respective user and includes personal data and historical data; and causing a display of a notification via a user device, wherein the notification has at least a portion of the plurality of health parameters and the generated at least one risk score.

Certain embodiments disclosed herein include a multifunctional body device (MBD), comprising: an electrocardiogram that includes at least three ECG leads; an impedance cardiography (ICG) system that includes at least two pairs of ICG electrodes; a body composition system that includes at least one bioimpedance electrodes; a calibration system that is configured to calibrate raw sensor data collected via the at least three ECG leads and the at least two pairs of ICG electrodes, wherein the calibration system includes reference calibration resistors; a network interface for observing network traffic.

### BRIEF DESCRIPTION OF THE DRAWINGS

The subject matter disclosed herein is particularly pointed out and distinctly claimed in the claims at the conclusion of the specification. The foregoing and other objects, features, and advantages of the disclosed embodiments will be apparent from the following detailed description taken in conjunction with the accompanying drawings.

FIG. 1A is an example network diagram utilized to describe the various embodiments.

FIG. 1B is a schematic diagram utilized to describe the various embodiments.

FIG. 2 is an example flowchart illustrating a method for determining user-specific risks according to an embodiment.

FIG. 3 is an example flowchart illustrating a method for determining a plurality of health parameters according to an embodiment.

FIG. 4 is a schematic diagram illustrating a top view of a multifunctional body-scale device (MBD) according to one embodiment.

FIG. 5 is a schematic diagram illustrating a three-dimensional view of a multifunctional body-scale device (MBD) according to one embodiment.

FIG. 6 is a schematic diagram illustrating a bottom view of a multifunctional body-scale device (MBD) according to one embodiment.

FIG. 7 is a schematic diagram illustrating contact points according to one embodiment.

FIG. 8 is a flow diagram illustrating a method of body weight evaluation according to an embodiment.

FIG. 9 is a flow diagram illustrating a method of buttock fat evaluation according to an embodiment.

FIG. 10 is a flow diagram illustrating a method of thigh fat evaluation according to an embodiment.

FIG. 11 is a flow diagram illustrating a method of visceral fat evaluation according to an embodiment.

FIG. 12 is a flow diagram illustrating a method of body temperature evaluation according to an embodiment.

FIG. 13 is a flow diagram illustrating a method of oxygen saturation evaluation according to an embodiment.

FIG. 14 is a schematic diagram of an evaluation system according to an embodiment.

### DETAILED DESCRIPTION

It is important to note that the embodiments disclosed herein are only examples of the many advantageous uses of the innovative teachings herein. In general, statements made in the specification of the present application do not necessarily limit any of the various claimed embodiments. Moreover, some statements may apply to some inventive features but not to others. In general, unless otherwise indicated, singular elements may be in plural and vice versa with no loss of generality. In the drawings, like numerals refer to like parts through several views.

The various disclosed embodiments provide a multifunctional body-scale device (MBD) configured to include one or more functionalities in a single MBD apparatus. The one or more functionalities may include, but is not limited to, bathroom scale, body compositions, bidet, smart body scale, fecal evaluation, urinalysis, pulse oximetry, electrocardiogrampedance cardiography (ICG), ambient condition monitoring, fingerprint scanning, satellite navigation or global positioning system (GPS), and the like, and any combination thereof. The MBD is configured to monitor a user and/or environment of the user by collecting data for at least one functionality and communicating collected data for analysis at a server. It should be noted that collecting of data may be performed without active participation of the user facilitating continuous monitoring of user and user environments, particularly relevant to health conditions of the user.

The embodiments disclosed herein collect sensor data from a plurality of sensors on the MBD. The plurality of sensors may include ECG leads, ICG electrodes, bioimpedance electrodes, temperature sensor, and the like, and more. It has been identified that analyses of collected sensor data, particularly for such plurality of sensor data, may create a large computational load that may not be practically performed by a device that collects such sensor. However, the disclosed embodiments communicate the collected sensor data to a separate system in, for example, a cloud environment, over a network. The versatile system in the cloud environment allows effective collection, processing, and storing of the sensor data from the plurality of sensors on the MBD. In an embodiment, a multi-layer neural network is trained and applied to the plurality of sensor data to determine values for the plurality of health parameters, for example and without limitations, ranging from cardiovascular stroke volume, fluid distribution, skeletal muscle mass, to temperature, and more.

It has been identified that measurements of multiple sensor data result in large noise and fluctuations from various environmental factors, thereby decreasing the accuracy of the collected sensor and determined health parameters. Particularly, collecting ICG signals and ECG signals have been identified to interfere with one another to create large noise signals. The disclosed embodiments apply at least one trained machine learning algorithm identify potential artifacts and noises and eliminate them for preprocessed, clean ICG and ECG sensor data. In addition, such ICG and ECG sensor data may be manipulated with a single timeline, which may be further processed to determine various cardiovascular and ECG-related health parameters such as, but not limited to, stroke volume, cardiac output, left ventricular end systolic and diastolic volumes, ejection fraction, total systolic and diastolic times, isovolumetric contraction and relaxation time, myocardial perfusion index, stroke volume variation, and the like, and any combination thereof.

The disclosed embodiments further provide a system and a method for personalized evaluation of data collected at the MBD. The MBD platform includes an external processing system for determining values for the one or more functionalities included in the MBD. The evaluation system applies at least one algorithm, such as a machine learning algorithm, to accurately and efficiently process the collected data. The processing of the collected data may be personalized based on user profiles. It should be appreciated that the MBD platform that includes an external processing system enables conservation of processing power and memory at the multifunctional body-scale device (MBD). Moreover, the complexity and size and the multifunctional body-scale device (MBD) may be maintained with reduced complexity and size.

In an embodiment, the risk assessment scores are uniquely determined for a user. The user profile data, historical data, and currently measured and collected plurality of sensor data are applied to the at least one algorithm. In an embodiment, the at least one algorithm may include, for example, but is not limited to, a classifier model. The at least one machine learning algorithm is trained against a training set from users from wide range of demographical, medical, socioeconomical, and the like background. It should be noted that the risk assessment scores generated according to the disclosed embodiments provide an accurate outlook of one or more potential risk events that are specifically determined for the user. It should be noted that the user-specific risk assessments are determined based on a large pool of health parameters (e.g., currently measured and determined and/or historical health parameters from previous measurements) of the user as well as various user profile data to provide a comprehensive determination of risks. Currently implemented techniques are not only determined for a general audience, that it not user-specific, but also determined based on few data points, for example, few measurements of one or two health parameters.

Moreover, it has been identified that health risks from subtle changes in health-related parameters are challenging to detect and often remain undetected until the later stages after much development of health conditions. In addition, current techniques often require specific procedures to be performed at a hospital, or the like facilities, that are not readily available or accessible. The disclosed embodiments utilize the MBD to enable facilitated and continuous monitoring of one or more physiological conditions (or health parameters). More particularly, the MBD collects user data (e.g., bioimpedance signals, ICG signals, ECG signals, etc.) in a non-invasive manner and allows for accurate determining of multiple health-related parameters (or physiological conditions) by utilizing at least one machine learning algorithm. The artificial intelligence-based detection algorithm utilizes user specific profile data (e.g., weight, ethnicity, gender, etc.) for detecting cardiovascular parameters and various body compositions (e.g., skeletal muscle mass, visceral fat mass, water mass, etc.) to account for user specific features and enable personalized evaluation of the collected data. For example, an identical signal reading from the MBD may result in different body composition values and/or cardiovascular parameter values based on the user specific data.

The MBD, of the disclosed embodiments, is capable of collecting additional data, such as, but not limited to, physiologic, psychologic, home, local environment, medical health records, and the like, and any combination from the user and/or external sources that are proximate to the MBD and/or user. Such additional data may include, but is not limited to, diet, mood, social media, user's medical record, population health data, and the like, and any combination thereof. Furthermore, in some embodiments an authorized caregiver or an authorized family member of the user may be allowed to override the data entered or collected from different sources and sensor(s).

The embodiments disclosed herein present a non-invasive multifunctional platform that efficiently and accurately determines the plurality of health parameters by combining one or more functionalities collected from MBD and processing at the external evaluation system. The combination of multiple functionalities in a single platform from collection to analyses enables comprehensive evaluation and determination of health risks. In addition, the each of the sets of sensor data (e.g., bioimpedance, ICG, ECG, etc.) may be processed to determine multiple health parameters rather than a single value for each set of sensor data. As an example, the bioimpedance signals are utilized to determine multiple body compositions such as, but not limited to, skeletal muscle mass, visceral fat, fluid distribution, and the like, and more. It should be noted that such shared sensor data amongst multiple functionalities (or health parameters) allow reduced complexity and form factor in the multifunctional device eliminating separate sensors for each and every functionality. To this end, the disclosed embodiments allow the conservation of cost, energy, and computing resources at the MBD platform as a whole.

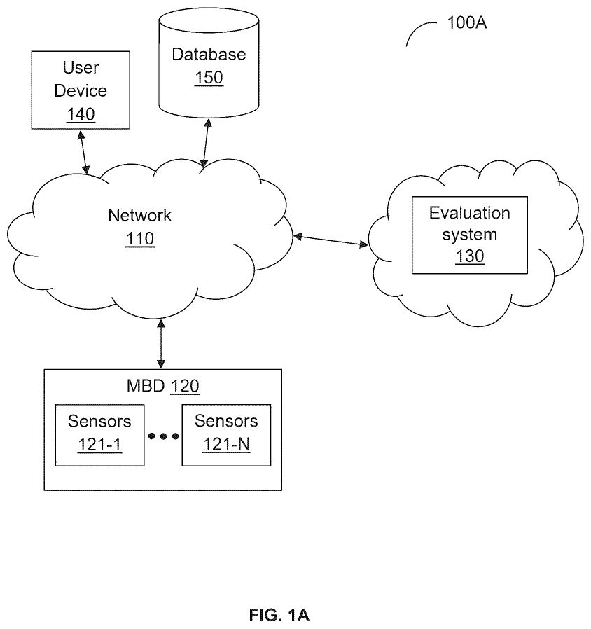

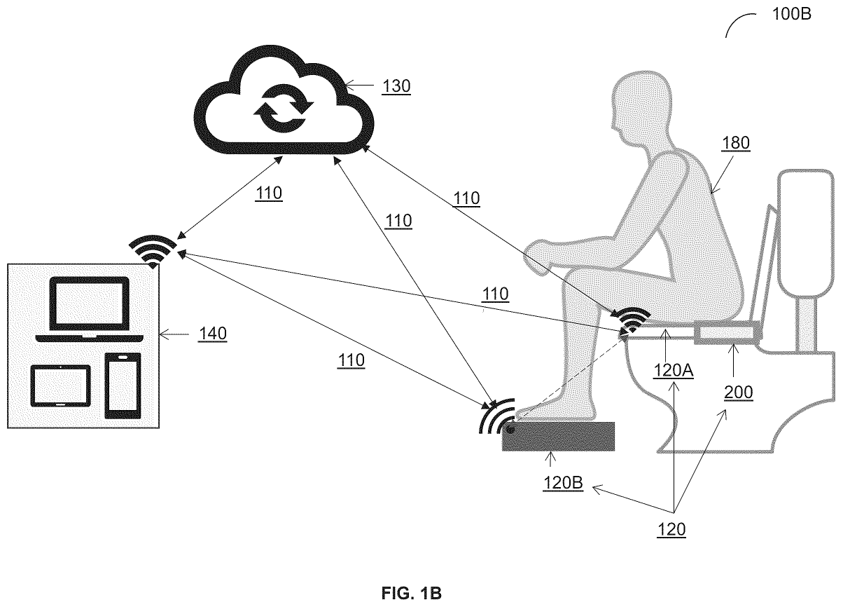

FIG. 1A shows an example network diagram 100A utilized to describe the various disclosed embodiments. In an example network diagram 100, a multifunctional body-scale device (MBD) 120, an evaluation system 130, a user device 140, and a database 150 are communicatively connected via a network 110. The network 110 may be, but is not limited to, a wireless, cellular or wired network, a local area network (LAN), a wide area network (WAN), a metro area network (MAN), the Internet, the worldwide web (WWW), similar networks, and any combination thereof.

The multifunctional body-scale device or simply multifunctional body device (MBD) 120 is a device configured to measure one or more device functionalities such as, but not limited to, bathroom scale, bidet, smart body scale, body compositions, fecal evaluation, urinalysis, pulse oximetry, electrocardiogrampedance cardiography, ambient condition monitoring, fingerprint scanning, satellite navigation or global positioning system (GPS), and the like, and any combination thereof. The MBD 120 collects data related to the device functionalities using one or more sensors 121-1 through 121-N (where N is an integer greater than 1, hereinafter referred to as sensor 121 and sensors 121). The sensors 121 include input devices for various device functionalities including, for example, but not limited to, detectors, microphones, temperature sensors, touch sensors, biosensors, bioimpedance sensors, electrocardiogramads, electro-optical sensors, electrochemical sensors, movement detector, camera (e.g., electromagnetic, IR, etc.), and the like. In some embodiments, the MBD 120 may include, for example and without limitations, electro-mechanical elements, display units, speakers, and the like. The components of the MBD 120 are further described herein below. The various sets (or types) or sensor data are collected from each of the sensors and communicated to the evaluation system 130 over the network. The collected sensor may be filtered or calibrated prior to transmitting to the evaluation system 130.

The evaluation system 130 is device, component, or the like, configured to receive data collected through at least one sensor 121 at the MBD 120 of a user, user excreta, ambient conditions, and the like, as well as user profile data. The evaluation system 130 determines a value (or state) for the evaluating functionality based on the data collected at the MBD 120.

The user profile for each individual user of the MBD 120 includes, for example, but not limited to, personal data, preferences, historical data, and the like, and any combination thereof. Personal data may include different information about the user ranging from, but not limited to, demographic information (e.g., age, height, weight, biological gender at birth, current gender identity, race, location, profession, work environment, living conditions, etc.), social information (e.g., work, family, hobbies, etc.), to medical information (e.g., medical conditions, allergies, dietary restrictions, medications, etc.). The user profile may include preferences for lifestyle, diet, and the like that may be defined by the user or authorized personnel as well as historical data of, for example, MBD measurements, determined values for evaluating functionalities, and the like, and more. As noted above, the evaluating functionality includes, for example, body weight, body fat distribution, skeletal muscle distribution, body compositions, fecal evaluation, urinalysis, oxygen saturation, electrocardiogrampedance cardiography (ICG), ambient condition monitoring, fingerprint scanning, satellite navigation or global positioning system (GPS), and the like, and more.

In an embodiment, the evaluation system 130 may be configured with an artificial intelligence (AI) engine (not shown) that can apply at least one algorithm, such as a machine learning algorithm, on user profile data, real-time measurements, feedback data, and the like, and more, to dynamically provide accurate evaluation of the data (or signals) collected and received from the MBD 120. The AI engine may be trained through a supervised model, an unsupervised model, or a combination of both to improve accuracy with continued usage of the MBD 120 and the evaluation system 130. The machine learning algorithms used for training may include, for example, neural networks, decision trees, k-nearest neighbor, support vector machine (SVM), and the like.

The evaluation system 130 is further configured to provide personalized evaluation of health conditions and/or risks that may be communicated to a user through, for example, a user device 140 via a graphical user interface (GUI). In a further embodiment, the evaluated data may be stored in a memory or in the database 150. It may be further understood that the evaluation system 130 may be a cloud computing platform. The cloud computing platform may be a private cloud, a public cloud, a hybrid cloud, or any combination thereof.

The user device 140 may be, but is not limited to, a personal computer, a laptop, a tablet computer, a smartphone, a wearable computing device, or any other device capable of receiving, processing, and displaying information and/or notifications. The user device 140 may be associated with individual users of the MBD 120 to interact with the MBD 120 and the evaluation system 130 through a graphical user interface (GUI) presented on the user device 140. In an embodiment, at least one user which includes, for example, each member of the household in which the MBD 120 is located, has access to the user device 140 to observe collected and evaluated data in the MBD 120 and the evaluation system 130. It should be noted that a plurality of user devices 140 may be allowed to access information collected by the MBD 120 as well as data analyzed at the evaluation system 130.

In some embodiments, the user device 140 includes mobile application for remote control of the MBD 120 and interaction of the MBD 120 and evaluation system 130 with the user. The mobile application is displayed to the user via a GUI and utilized to build the user profile by capturing user information, which may be input by the user (via the user device 140) and/or captured by the MBD 120. The user profile may include, for example, but not limited to, personal data, preferences, historical data, and the like, and any combination thereof. Personal data may include different information about the user ranging from demographic information (e.g., age, height, weight, biological gender at birth, current gender identity, race, location, profession, work environment, living conditions etc.), social information (e.g., work, family, hobbies, etc.), to medical information (e.g., medical conditions, allergies, dietary restrictions, medications, etc.). In addition, the user profile may include, for example dietary preferences (e.g., omnivore on a keto diet, does intermittent fasting, etc.), dietary pleasures (e.g., binging on beer or chocolate ice cream, and the like), fitness or characteristic strengths, usual moods, trigger points, and the like and more.

The mobile application may be utilized to set health and wellbeing goals for individual users. As an example, aspirational identity can be established to include an avatar and a short phrase capturing the user's vision (e.g., I am a runner who wants to stay healthy and fit and keep running as long as possible), values related to lifestyle (e.g., I eat only sustainably harvested organic food or I explore the world going from places to places), and social identity.

In some implementations, the large language models (LLMs) may be employed for generating notifications to a user via the user device 140. The LLMs are applied to user profile data (e.g., personal data, historical data, medical data, etc.) and currently analyzed health parameters for the respective used to generate notifications including, for example, but not limited to, information, support, recommendation, and assistance to the specific user. It should be noted that the generated notification provides unique information that are relevant to the specific user that assistance navigation through the identified health risks. In an example embodiment, the LLMs are employed to generate series of questions. The notification and questions may be caused to be displayed at a user device 140 through as GUI.

Additional data regarding daily activities may be captured or input into the mobile application deployed at the user device 140. The captured daily activities such as, but not limited to, mood, emotion, feelings, trigger points and reflection, forgiveness and gratitude, highlights of the day, dietary and hydration activity, at least one temperature reading of surrounding, and the like, and any combination thereof, may be captured automatically and/or manually. In an example embodiment, the captured data may be analyzed further to conduct group analyses indicating user's condition compared against individuals that belong to similar cohort. In another example embodiment, the mobile application may be configured to engage with other individuals related to the specific user, for example, but not limited to, family, community, expert care team members, health professionals, to share information and provide needed support for the user of the MBD platform.

The data captured at the mobile devices may be included in the user profile data for further analyses of user conditions related to their physical and mental wellbeing. In an embodiment, at least portions of the user profile data are stored at the database 150 and retrieved by the evaluation system 130 for personalized evaluation and personalized recommendations for individual users, which may be provide to the user via a GUI on the user device 140.

The database 150 may be part of the evaluation system 130 or may be separate and communicatively connected via the network 110. The database 150 is utilized to store, for example, data collected from sensors 121 of the MBD 120, evaluated data (and values) at the evaluation system 130, as well as user profiles for individual users of the MBD 120.

FIG. 1B is an example schematic diagram 100B of the various disclosed embodiments of the multifunctional body-scale device (MBD) platform according to an example embodiment. For simplicity and without limitation of the disclosed embodiments, FIG. 1B will also be discussed with reference to elements shown in FIG. 1A. In the example diagram 110B, a toilet seat 120A including a toilet seat control 200, a base pad footrest 120B, an evaluation system 130, and at least one user device 140 are communicatively connected via a network 110. The toilet seat 120A, toilet seat control 200, and the base pad footrest 120B are components of a MBD 120. A user 180 may utilize the MBD 120 where, in an embodiment, such utilization can trigger collection of measurements for at least one device functionality at the MBD 120.

It should be noted that the disclosed embodiments are described with respect to a toilet seat and the base pad footrest for illustrative purposes and does not limit the scope of the various disclosed embodiments described herein. The MBD 120 may be configured to include one or more components, different combination of components, and the like, without departing the scope of the various disclosed embodiments described herein.

According to the example embodiment, the MBD 120 includes components, such as, but not limited to, the toilet seat 120A, a toilet seat control 200, and the base pad footrest 120B, where the toilet seat control 200 is an extended component of the toilet seat 120A that the user may hold or touch using their hands. Each of the components of the MBD 120 includes one or more sensors for measuring device functionalities. In an embodiment, each component, the toilet seat 120A and the base pad footrest 120B, is configured to communicatively connect to at least one user device 140 and/or the evaluation system 130 over a network 110. As noted above, the MBD 120 collects data from, for example, but not limited to, the user 180, excreta of the user 180, ambient conditions in the vicinity of the MBD 120, and the like, and transmits the collected data to the user device 140 and/or the evaluation system 130. The evaluation system 130 may store collected data and perform advanced data management and analysis. In some embodiments, analysis for device functionalities may be duplicated in the evaluation system 130 and the user device 140. In an embodiment, the MBD 120 may include pairs of free-form electrodes that are not attached to, or part of, a particular component or structure. As an example, the pairs of electrodes may be electrodes that may be attached to different parts of the body (e.g., arm, neck, abdomen, etc.). Such free-form electrodes may be used independently or in conjunction with one or more components of the MBD 120.

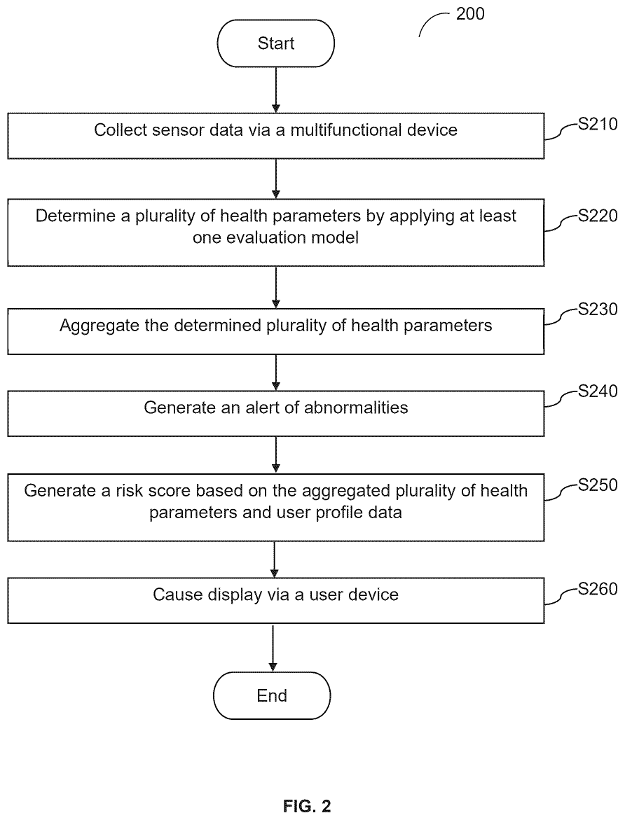

The toilet seat 120A is a hinged or attached unit consisting of a round or oval open seat, sometimes with a lid, and is bolted onto the bowl of a toilet. The toilet seat 120A includes the seat itself, which may be contoured for the user 180 to sit on, and an optional lid, which covers the toilet when it is not in use. In an embodiment, the toilet seat 120A includes one or more sensors, for example, but not limited to, electromagnetic sensors, capacitive sensors, inductive sensors, electrodes, pressure sensors, and the like, and any combination thereof. The toilet seat 120A is adapted to measure a plurality of device functionality simultaneously. In a further embodiment, the toilet seat 120A houses an electromechanical system for delivering water and air at desired conditions, for example, temperature, pressure, and direction. In some embodiments, excreta (e.g., fecal and/or urine) collected in the toilet may be analyzed by, for example, but not limited to, an electromagnetic camera, a robotic biosensing assembly, and the like, configured at the toilet seat 120A. The toilet seat 120A may further include satellite navigation (SatNav) system and sensors that assess location and ambient conditions, respectively, of the toilet seat 120A. The toilet seat 120A is discussed in further detail herein below with respect to FIGS. 2-4.

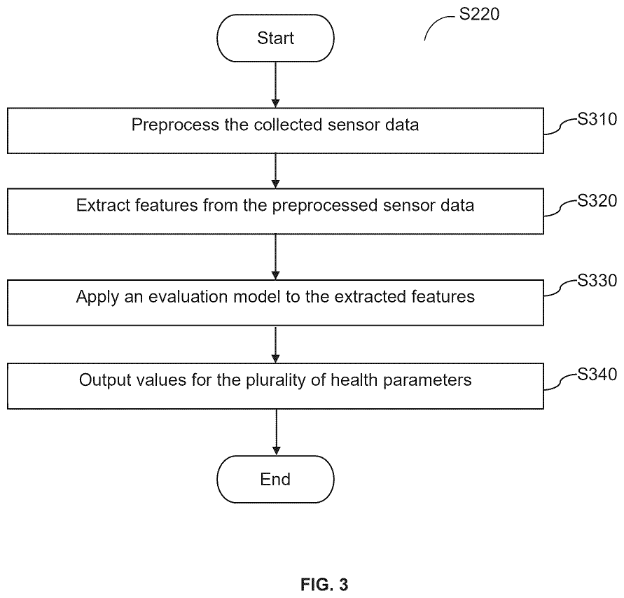

The base pad footrest 120B is placed on a surface close to the foot and provided for the user 180 to lay their feet and achieve a favorable posture while using the toilet. In an embodiment, the footrest 120B includes one or more sensors, for example, but not limited to, electromagnetic sensors, capacitive sensors, inductive sensors, electrodes, pressure sensors, and the like, and any combination thereof. In a further embodiment, the footrest 120B includes a satellite navigation (SatNav) system and sensors to assess the location and ambient conditions, respectively, of the footrest 120B. The base pad footrest 120B may include a heating system to maintain desired temperature at the surface. In an embodiment, the footrest 120B is connected to the toilet seat 120A for electric power and data communications. In another embodiment, the footrest 120B is independently powered and connected over the network apart from the toilet seat 120A. The footrest 120B may be selectively used when the user uses the MBD 120 based on, for example, user preference, need, status of MBD 120 and/or footrest 120B, and the like, and any combination thereof. The base pad footrest 120B is discussed in further detail herein below with respect to FIGS. 2 and 3.

In an example embodiment, the MBD 120 may include a base pad footrest 120B with a plurality of sensors for detecting multiple functionalities. In such case, the base pad footrest 120B may be configured to communicate with at least one of a user device 140 and an evaluation system 130 over a network to send collected data and further analyze for various functionalities. In a further embodiment, the MBD 120 with a base pad footrest 120B may be electrically and communicatively connected to other electrodes in various form factors, for example, but not limited to, free-form, hand-held handles, arm band, neck band, forceps, patches, pads, and the like, and any combination thereof, that includes sensors (or electrodes) to collect data related to a user 180 and/or the environment. In a further example embodiment, the other electrodes in various form factors may be directly connected to the user device 140 and/or evaluation system 130.

In an embodiment, the MBD may be a device with multiple free-form sensors. The sensors are electrodes that are electronically connected to the MBD and configured to collect bioimpedance measurements for multiple body composition parameters. In a further embodiment, the MBD includes four electrodes for bioimpedance measurements and at least three additional sets of electrodes for ECG measurements. In an example embodiment, the four sensors may be affixed as two neck-torso pair where each pair of sensors has one sensor at the neck and one sensor at the torso on opposite sides of the body. In a further example embodiment, sensors may be affixed to a forearm of a person, which may be utilized separately, or in combination with the neck-torso sensors. It should be noted that other configurations of sensor placement may be implemented to collect sensor data such as, but not limited to, bioimpedance, ECG, ICG, and more, from a user's body.

Electrocardiogramsor data is evaluated to analyze user cardiac condition. The electrocardiogramecorded by electric potential changes occurring between electrodes placed on a patient's torso to demonstrate cardiac activity. The ECG signal tracks heart rhythm and many cardiac diseases, such as poor blood flow to the heart and structural abnormalities. However, conventional caliper process requires skills and experiences. Further, since different pregnant women gain and keep fats in their bodies in different ways, results of measurements of their body fats vary. To this end, the disclosed embodiments utilize bioelectric impedance techniques to measure percent body fat or a total body fat, for example, as described above, for estimating the cardiovascular parameters (e.g., flow time, stroke volume, cardiac output, etc.).

In an embodiment, the ECG system, the ICG system, and bioimpedance system may be configured on a single system on chip (SoC) or in separate chip systems. In a further embodiment, the different systems of the MBD may be integrated into a printed circuit board (PCB). The ECG, ICG, and bioimpedance system may be different components within a larger system. In an embodiment, the MBD may include a calibration system that is configured to calibrate raw sensor data collected from one or more sensors (e.g., the ECG and ICG sensors. The calibration system may include reference calibration resistors. In a further embodiment, the MBD includes a power management unit for managing power in the device. The power management unit is configured to automatically switch to battery power from the main power supply during an outage. In addition, the power management unit monitors charge and discharge of the battery as well as the temperature of the battery for safety. A bus is configured to allow the different systems (e.g., ECG system, ICG system, body composition system, calibration system, etc.) to communicate.

FIG. 2 is an example flowchart illustrating a method for determining user-specific risks according to an embodiment. The method described herein is performed by an evaluation system 130, FIGS. 1A and 1B. In an embodiment, the evaluation system 130 is connected to a MBD 120, FIGS. 1A and 1B, which collects data from a user (e.g., a patient), over a network. The evaluation system 130 may be deployed in a cloud environment such as a private cloud, a public cloud, or a hybrid cloud. Examples of cloud environment includes, for example and without limitation, Amazon Web Service® (AWS), Microsoft Azure®, Google Cloud Platform® (GCP), or the like. The method of FIG. 3 is generally described for sensor data collected at a specific time frame for a specific user. It should be noted that the user-specific risks are personalized risks for a unique user based on the user's physiological conditions.

At S210, sensor data are collected. The sensor data are collected via plurality of sensors at an MBD (e.g., the MBD 120, FIG. 1A and FIG. 1B). In an embodiment, the sensor data may be collected for a predetermined time period by placing the plurality of sensors on a body surface of a target user. The sensor data includes multiple sets of sensor data such as, but not limited to, a bioimpedance measurement, ICG, ECG, weight, pulse oximetry, fingerprint scanning, and the like, and any combination thereof. In an embodiment, the sensor data may be collected from the surrounding environment for, for example, ambient condition monitoring, satellite navigation, GPS, and the like, and any combination thereof. In some configurations, sensor data for fecal evaluation, urinalysis, and the like, may be collected via the MBD.

In an embodiment, a tetrapolar electrode configuration (i.e., four electrodes) is employed for ICG signal (or ICG sensor data) collection. In an example configuration, two electrodes are placed on a neck, for example, on a sternocleidomastoideole, and two electrodes are placed on a lower chest or upper abdomen of a user's body. The configuration allows subtraction techniques to minimize extra-thoracic impedance changes, thereby collecting more accurate ICG signals. It should be noted that other configurations may be utilized without departing the scope of the disclosed embodiments.

In an embodiment, a 3-lead ECG configuration is employed to detect electrical activity of the heart of the user at different angles and to collect as ECG sensor data. As an example, the ECG leads may be placed on the right upper arm, left upper arm, and left lower leg. In a further embodiment, additional ECG leads may be utilized at other parts of the body.

In an embodiment, the bioimpedance signals (or bioimpedance sensor data) are collected via a bioimpedance spectroscopy (BIS) using multi-frequency electrodes. The bioimpedance signals are utilized to determine various body compositions such as, but not limited to, fluid level, water level, inter and intra cellular water, fat mass, muscle mass, and the like, and more. In an example embodiment, the BIS electrodes are placed on wrists and ankles, and the torso of a user. In an embodiment, the electrodes may be different types including, for example, but not limited to, gel electrodes, Transcutaneous electrical nerve stimulation (TENS) electrodes, and metal-based electrodes.

It should be noted that the various sensor data may be collected from different MBD arrangements in, for example, free-form electrodes, integrated form, toilet seat form, footrest form, or more. One of ordinary skill in the art would understand that the arrangement of MBD does not limit the scope of the disclosed embodiments.

In an embodiment, the collected sensor data are calibrated at the MBD prior to communicating with the evaluation system (e.g., the evaluation system 130, FIG. 1A). The MBD incorporates reference calibration resistors with specific resistance and reactance values that are used as controls during the calibration process. The controls ensure that the acquired data reflect the target modality of the various sets of sensor data. It should be appreciated that the calibration at the MBD provides stable and consistent calibration that are independent from the evaluation system or other systems connected over the network.

At S220, a plurality of health parameters is determined. A plurality of evaluation models is applied to the collected sensor data. The evaluation model may be an AI-based algorithm such as, but not limited to, multi-layer neural network, and the like, to determine values of the plurality of health parameters. In an embodiment, the evaluation model is separately applied to a set of collected sensor data such as, but not limited to, ICG, ECG, bioimpedance, and the like, and any combination thereof. The processing of the collected sensor data is further described herein below in FIG. 3 and FIGS. 8-14. In an embodiment, processing each set (or type) of sensor data allows determination of at least one health parameter. As an example, the analysis of ICG sensor data determines hemodynamic parameters, for example, but not limited to, stroke volume (SV), heart rate (HR), cardiac output (CO), ventricular ejection time (VET), and the like, and more.

At S230, the values determined plurality of health parameters are aggregated. The aggregated health parameters may be added to a memory and/or database (e.g., the database 150, FIG. 1A) in association with the specific user. Metadata such as, but not limited to, user ID, time stamp, and the like, and any combination thereof, may be associated with the determined health parameters and stored together. Some example health parameters may include cardiometabolic parameters such as, but not limited to, ventricular volume, ejection fraction, contraction, heart rate, stroke volume, vascular resistance, and the like, and more; and metabolic compositions such as, but not limited to, fat mass, water mass distribution, muscle mass, and the like.

In an embodiment, the aggregated health parameters may be analyzed to determine a trend of the health parameters. The trend may be determined based on time, progress of treatment, and the like, and more. In an example embodiment, the trend is tracking of one or more health parameters across different cycles at a given point of time, over an extended time frame, or the like, and more. It should be noted that the trend analysis allows effective monitoring of patient condition and treatment effectiveness.

At S240, an alert of abnormalities is generated. The alert, or notification, is generated upon detecting at least one abnormality in the aggregated plurality of health parameters. As an example, inconsistent heart rates cause generation of an alert. In another example, a low ventricular volume causes generation of an alert. In an embodiment, the abnormality may be determined by comparing to, for example, standard values, previous values of the parameters, consecutive values detected within the predetermined time period, and the like, and any combination thereof. In an embodiment, the alert is caused to be generated via a user device (e.g., the user device 140, FIG. 1A). In an embodiment, a report that describes the trend (e.g., status, change, etc.) of the plurality of health parameters is generated. The generated report is caused to be displayed via a user device (e.g., the user device 140, FIG. 1A).

At S250, a risk assessment score is generated for the user. The risk assessment score (or simply a risk score) predicts a risk probably for the user at different time frame, for example, but not limited to, at 72 hours, at 6 months, at 10 years, and the like, and more. In an embodiment, the risk score may be determined for different events such as, but not limited to, coronary death, fatal stroke, first occurrence of nonfatal myocardial infarction (MI), first occurrence of stroke, and the like, and any combination thereof.

A risk assessment model is applied to the determined plurality of health parameters, user profile, and the like to determine the risk assessment scores. The user profile is unique for each user and includes personal data, preferences, historical data, and the like, and any combination thereof. The user profile may be retrieved from a database (e.g., the database 150, FIG. 1A). The risk assessment model may be at least one algorithm such as, but not limited to, classifiers (e.g., support vector machine (SVM), random forest, etc.), artificial neural networks (ANNs), eXtreme Gradient Boosting (XGBoost), Light Gradient-Boosting Machine (LightGBM), and ensemble models, and the like, and any combination thereof. In some implementations, the risk assessment model may be analyzed against reference model outlined as per World Health Organization (WHO) cardiovascular disease (CVD) Risk assessment. In an embodiment, the risk assessment scores for the user may be associated with metadata such as, used ID, timestamp, and the like, and any combination thereof, and stored in a memory or a database (e.g., the database 150, FIG. 1A).

In an embodiment, the plurality of health parameters are retrieved from the database for generation of risk assessment scores. It should be noted that applying the generated health parameters, for example, historical health parameters, that are readily available, allows rapid processing and determination. That is, repeated processing of the multiple sensor data are eliminated to reduce processing time and computing power.

Personal data may include different information about the user ranging from demographic information (e.g., age, height, weight, biological gender at birth, current gender identity, race, location, profession, work environment, living conditions etc.), social information (e.g., work, education, family, hobbies, etc.), to medical information (e.g., medical conditions, allergies, dietary restrictions, medications, etc.). In addition, the user profile may include, for example dietary preferences (e.g., omnivore on a keto diet, does intermittent fasting, etc.), dietary pleasures (e.g., binging on beer or chocolate ice cream, tobacco use, alcohol consumption, and the like), fitness or characteristic strengths, usual moods, lifestyle habits, trigger points, and the like and more. The historical data may include previously determined values for the health parameters, physician assessment of the cardiovascular risk category (previous and current), and the like, and any combination thereof.

The risk assessment model is trained with training datasets including diverse populations (e.g., age, ethnicity, gender, lifestyles, and the like, and more) to develop an accurate and unbiased model. The risk assessment model performances are tested using performance metrics such as, but not limited to, fairness and bias (e.g., LIME and SHAP), interpretability, generalizability (e.g., L1/L2 regularization technique), and more. In addition, the risk assessment model may be further trained with feedback data received on the output risk scores.

According to the disclosed embodiments, the risk assessment scores are determined for a specific user in account of various sections of the user including their sociodemographic, habits, physiological conditions, and the like, from their user profile, as well as their current health parameters as determined using the MBD. It should be noted that the risk assessment model allows a comprehensive analysis of the user that is not limited to the sensor measurements. As an example, risk scores for two patients with similar physiological conditions in body composition, CVD, and the like, but are different ethnicities residing in different geographical locations may be uniquely determined using risk assessment model. It should be noted that current techniques lack such comprehensive analysis, and such two patients may not be distinguishable.

At S260, the risk assessment scores are caused to be displayed via a user device. The user device (e.g., the user device 140, FIG. 1A) may be associated with a user (being measured), a physician, or both. In an embodiment, a notification may include, at least in part, for example, but not limited to, user profile data, the determined health parameters, risk assessment scores, and the like, and any combination thereof. The information in the notification may differ depending on the audience. For example, a user may receive a notification with an easy-to-understand explanation of health conditions, educational resources to improve understanding of health conditions, and cardiac risk factors, personalized recommendations, and the like, and more. In another example, a physician may receive a notification including medical data with visualization and interpretation, efficient workflow for user and risk assessment, comparison between historical and current trends, and the like, and more.

In an embodiment, a graphical user interface (GUI) may be employed for user and/or physician interaction with the notification via the user device. The GUI provides, for example, interactive analysis tools, visual alerts or statuses, visualized representation of body compositions, and the like, and more.

In an embodiment, a large language model (LLM), or like language models, is employed to generate and cause display of notification to the specific user via a user device. The notification may include, for example, but not limited to, suggestions, recommendations, a series of questions, and the like, and more, to provide assistance to the individual to navigate the identified health risks.

FIG. 3 is an example flowchart S220 illustrating a method for determining a plurality of health parameters according to an embodiment. The method described herein is performed in the evaluation system 130, FIG. 1A.

In an embodiment, sets of sensor data received from the MBD (e.g., the MBD 120, FIG. 1A) include, for example, but not limited to, ICG, ECG, bioimpedance, and the like measurements, and any combination thereof. In an embodiment, the ICG and the ECG sensor data are collected concurrently for a predetermined time period. In a further embodiment, the bioimpedance measurement is collected at a separate time, for example, right after the predetermined time period for collecting ICG and ECG sensor data.

At S310, the collected sensor data are pre-processed. The sets of sensor data include, for example, ICG, ECG, and the like signals, which are processed separately. In an embodiment, the ICG and ECG signals are collected simultaneously via the MBD using respective electrodes. In an embodiment, the bioimpedance measurements for body composition analyses (BCA) are collected subsequent to the ICG and ECG signals.

It has been identified that concurrent measurements of ICG and ECG signals are challenging due to the interference between the signals. To this end, noisy and inaccurate signals (or sensor data) are obtained from both ICG and ECG electrodes. In an embodiment, pre-processing techniques are employed to remove artifacts, reduce noise, and enrich the data, thereby generating accurate sensor data for ICG and ECG.

In an embodiment, artifacts from, for example, breathing, movement, pressure, and the like, and any combination thereof, are removed for the collected sensor data. In an embodiment, the removal is performed immediately after collection. An algorithm such as, but not limited to, an AI model, machine learning model, and the like, is utilized to identify and classify the artifacts based on the type of artifact (e.g., breathing, movement, posture, sensor interference, interference from other signals, etc.). In an embodiment, the identified artifacts from voluntary or involuntary movements are eliminated. In a further embodiment, sensor data from other sensors in the MBD may be utilized to identify and correct the artifacts, postures, and movement variations. It should be noted the pre-processing using at least one machine learning algorithm effectively removes distortions and interferences between signals effectively and accurately to allow concurrent analysis of ICG and ECG data.

In a further embodiment, the contextual data enrichment may be performed. For the ICG sensor data, additional data stream that represents a first derivative of the signal may be generated. Contextual tags (or IDs) that describe potential artifacts, anomalies, or other physiological events, are added to the relevant data stream to enrich and provide context to the collected sensor data. The identified artifacts may be labeled on the ICG sensor data and the ECG sensor data.

In an embodiment, cleaned sensor data from which identified artifacts are removed are represented as signal plots for each of the ECG and ICG. The cleaned ECG data and the ICG data are synchronized and presented as a single temporal scale. For example, the cleaned ECG data and the ICG data are represented as an overlay of two plots that are synchronized in time (aligned with respect to a same timeline), which are utilized for further analysis of one or more health parameters. It should be noted that the synchronous representation and analysis of the ECG and ICG data provide clinical insights that may be otherwise not attainable. Moreover, it should be noted that such simultaneous detection under synchronous time is not attainable without the disclosed embodiments described herein.

The pre-processed sensor data of ECG and ICG data may be generated in various formats enabling effective integration into other systems such as existing clinical monitoring systems. The versatility of processing ECG and ICG data enables adaptive and seamless integration of cleaned sensor data into various and multiple healthcare facilities.

At S320, features of the pre-processed data are extracted. At least one algorithm such as a machine learning algorithm is applied to the pre-processed sensor data of the ECG and/or the ICG to extract notable features. The notable features are determined from the ECG and ICG plots. Some examples of extracted features include, for example, but not limited to, waveform characteristics, derived parameters, time-domain features, frequency-domain features, and the like, and any combination thereof. The waveform characteristics analyze the shapes of ECG and ICG waveforms, such as peak amplitude, rise time, and overall morphology. The derived parameters determine additional parameters based on the combined data, such as systemic vascular resistance. The time-domain features provide timing information such as, duration of various cardiac cycle phases (e.g., pre-ejection period, LVET), and intervals between ECG waves (e.g., PR interval, QRS duration, etc.). The frequency-domain features relevant to cardiac function are identified through spectral analysis of the impedance waveforms. In an embodiment, features are extracted for each cycle of the ICG and ECG sensor data, which may include a plurality of cycles representing the plurality of heart beats that are monitored during the predetermined time period for collecting the sensor data. In an example embodiment, the analysis of synchronous ICG and ECG sensor data at multiple cycles provide broader insights, for example, detection of arrhythmic events.

At S330, an evaluation model is applied to the extracted features. At least one evaluation model is applied to a set of sensor data (e.g., the cleaned ICG data, the cleaned ECG data, and the bioimpedance data). The evaluation model is a multi-layer neural network that includes multiple hidden layers that extract hierarchical features to determine values for the plurality of health parameters that are derived from and associated with the sensor data. In an embodiment, the extracted features are input into the evaluation model. In an embodiment, other cardiovascular-related parameters such as, but not limited to, blood pressure are measured or retrieved from the database and added as input data.

The evaluation model is configured to determine various cardiovascular monitoring (or hemodynamic monitoring) values for parameters related to ECG and ICG monitoring including, for example, timing of cardiac cycle phases, stroke volume, ventricular volumes, heart rate variability, cardiac output, pre-ejection period (PEP), left ventricular ejection time (LVET), wave morphology and intervals, beat-to-beat analysis, variability in feature trends, maximum and minimum values per heart beat cycle, area under the curve of the signal, signal derivatives, augmentation index, and the like, and more. In addition, the evaluation model is configured to determine values for body composition parameters such as, but not limited to, fat mass, water mass distribution, muscle mass, and the like, using bioimpedance data. In an embodiment, further analyses may be performed to determine pulmonary function based on cardio metabolic data, and kidney function based on metabolic, cardiac, and socio-demographic data.

At S340, values for each of the plurality of health parameters are output. The values for the plurality of health parameters that are output from the at least one evaluation model. In an embodiment, a data structure is generated to include the values for the plurality of health parameters for the user for the specific measurement session. The data structure include plurality of fields for the plurality of health parameters. The output data structure may be associated with metadata such as, but not limited to, user ID, time and date stamp, location ID, session ID, and the like, and any combination thereof. In an embodiment, the output data structure of the plurality of health parameters are stored in a database (e.g., the database 150, FIG. 1) and may be referred back as historical data for the specific user.

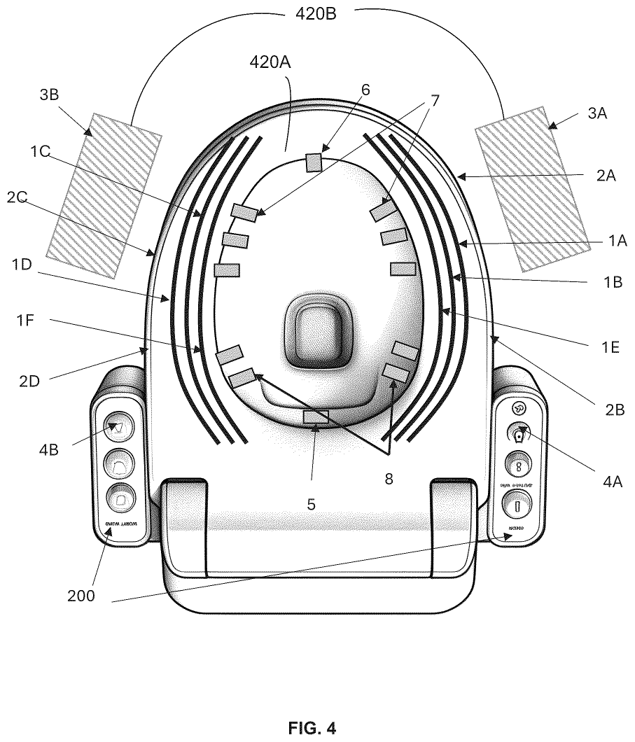

FIG. 4 is an example schematic diagram of a top view of the MBD according to one embodiment. In addition to the two components of the MBD, the toilet seat 420A and the base pad footrest 420B, introduced in FIG. 1B, a toilet seat control 200 is shown in the top view of the MBD. The toilet seat control 200 is one or more extensions from the toilet seat 420A utilized for operation of the MBD. In the example diagram of FIG. 4, two toilet seat controls 200 are shown on either side of the toilet seat 420A, each including a control button, 4A and 4B, for operating the MBD through contact with, for example, a finger of a user. In an example embodiment, the control buttons 4A and 4B may be electrodes.

The toilet seat 420A includes one or more pairs of sensors (e.g., 1A and 1F, 1B and 1C, and 1D and 1E) to measure device functionalities from direct contact with the user's body part such as, but not limited to, buttocks, thighs, and the like, and any combination thereof. In an example embodiment, a pair of body mass evaluation sensors on the toilet seat 420A are utilized to determine body mass, as well as body fat distributions, and more, by taking bioelectric impedance measurements.

In another example embodiment, a pair of temperature sensors on the toilet seat 420A surface measures a user's body temperature through contact. In addition, the pair of temperature sensors may measure ambient temperatures of the environment surrounding the toilet seat 420A. The temperature sensors may be at least one of NTC (negative thermal coefficient) thermistor, complementary metal-oxide-semiconductor (CMOS) analog temperature sensor, ultraviolet sensor, infrared sensor, and the like. In some embodiments, a plurality of pairs of sensors may be utilized for measuring a single device functionality. The one or more pair of sensors may detect other ambient conditions, such as, but not limited to, humidity, pressure, and the like, of the surroundings of the MBD and/or the user. In an embodiment, the one or more sensors may be, for example, electromagnetic sensors, capacitive sensors, inductive sensors, and the like, and electrodes capable of sensing signals ranging from, for example and without limitation, 0.5 mV to 1.5V and 0.5 Hz to 1 KHz.

The toilet seat 420A further includes one or more contact points, 2A, 2B, 2C, and 2D, that collect weight inputs for a user using the MBD 120. Such contact points 2A, 2B, 2C, and 2D are each associated with a pressure transducer which converts projected weight at each of the points into a corresponding electrical signal and sends the signal to the evaluation system. The evaluation system converts the electric signal from each of the contact points 2A, 2B, 2C, and 2D, for example, separately and/or collectively, into a projected weight of the user. In an embodiment, the electrical signal indicating projected weight may be sent to the user device 140. In an example embodiment, the pressure sensitive transducers are located at the bottom side of the toilet seat 420A.

In a further example embodiment, the toilet seat 420A includes components of a bidet (e.g., 5 to 8), which is a plumbing fixture or a type of a sink for washing user body parts, such as, the genitalia, perineum, inner buttocks, anus, and the like. At least one bidet nozzle 7, 8 of the bidet that is similar to that on a kitchen sink sprayer delivers a spray of water to assist in anal cleansing and cleaning the genitals after defecation and urination. The bidet may be mechanical or electro-mechanical. It should be noted that electromechanical system of the toilet seat 420A is utilized for delivering water and air at desired temperature, pressure, direction, and more.

In an embodiment, the toilet seat 420A further includes an electromagnetic camera 6 that triggers a flashlight and captures the image of the excreta (e.g., fecal, urine, etc.) inside the toilet bowl. A robotic biosensing assembly is also included in the toilet seat 420A for collecting the urine sample and analyzing different substances (analytes) of the excreta. It should be noted that such electromagnetic camera and/or robotic biosensing assembly may be configured to monitor the degree of usage, for example, number of times used, types of excretion detected, and the like, and any combination thereof for each user, group of users, general tracking, and the like, and any combination thereof.

The base pad footrest 420B of the MBD includes contact points, 3A and 3B. The contact points 3A and 3B collect measurements for at least one device functionality when the soles of the user's feet come in contact. Each contact point, 3A or 3B, includes a set of sensors such as, but not limited to, temperature sensor, impedance-based body mass evaluation sensors, and the like, and any combination thereof. As noted above, temperature sensors detect user body temperature, ambient temperature, or both. The contact points may further include sensors for assessing other ambient conditions such as, without limitation, humidity, pressure, and more. In an embodiment, the sensor is, for example, but not limited to, electromagnetic sensors, capacitive sensors, inductive sensors, and the like, and electrodes capable of sensing signals ranging from, for example and without limitation, 0.5 mV to 1.5V and 0.5 Hz to 1 KHz.

The base pad footrest 420B further includes, without limitation, a satellite navigation system to assess the location and a heating system to maintain desired surface temperature of the base pad footrest 420B.

It should be noted that the disclosed embodiments of FIG. 4 are described with certain type, number, location of sensors for illustrative purposes and do not limit the scope of the various disclosed embodiments described herein. It should also be noted that the disclosed components of the MBD may be utilized independently or in any combination to perform various device functionalities.

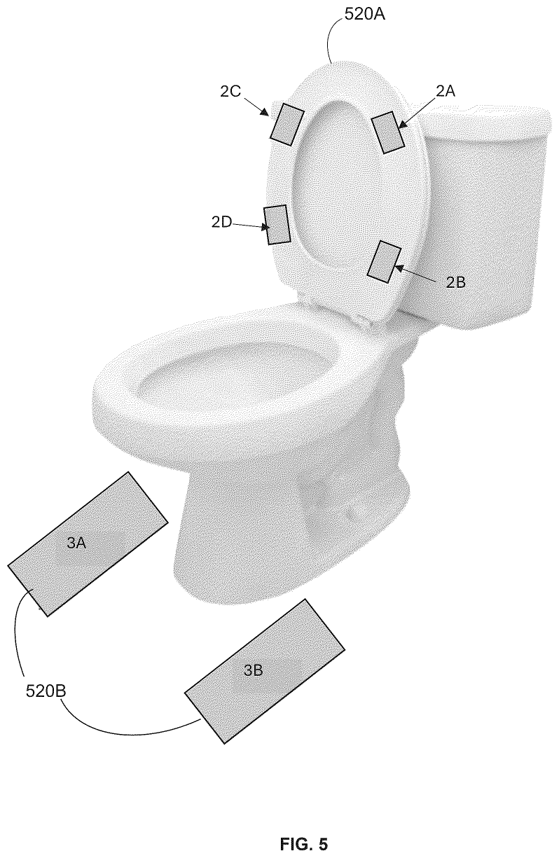

FIG. 5 is an example schematic diagram of the MBD in a toilet seat and foot rest configuration according to one embodiment. The pressure transducers associated with the contact points 2A through 2D are located at the bottom side of the toilet seat 520A. In the example embodiment, the toilet seat 520A may be attached to, for example, a toilet bowl using a hinge at one end of the toilet seat 520A, in this case, the end close to the contact point 2B. In such configuration, the toilet seat 520A may be swung up or down with respect to the hinge as illustrated using the arrows. The top view of the base pad footrest 520B illustrates the contact points 3A and 3B on the top surface of the base pad footrest 520B.

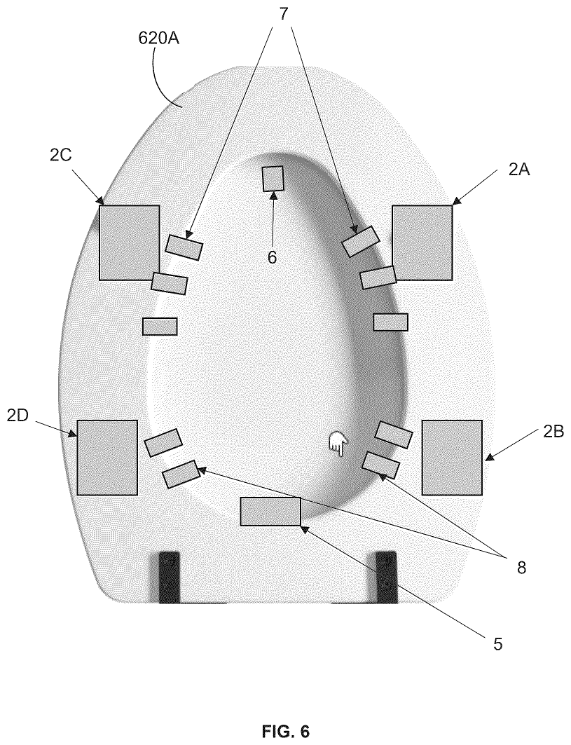

FIG. 6 is an example schematic diagram of a bottom view of the toilet seat 620A of the MBD according to one embodiment. The bottom view of the toilet seat 620A illustrates the pressure transducers, 2A, 2B, 2C, and 2D, associated with the respective contact points described in FIG. 4 (e.g., contact points 2A, 2B, 2C, and 2D). The pressure transducers collect electrical signals and/or changes in the electrical signal when the user uses the MBD (and the toilet) and contacts the toilet seat 620A at, at least one contact points associated with the pressure transducers 2A, 2B, 2C, and 2D. In an embodiment, the electrical signals are utilized in body weight evaluation to determine the user's body weight. The bottom view in FIG. 6 further illustrates the components of the bidet 5 through 8 on the example toilet seat 620A.

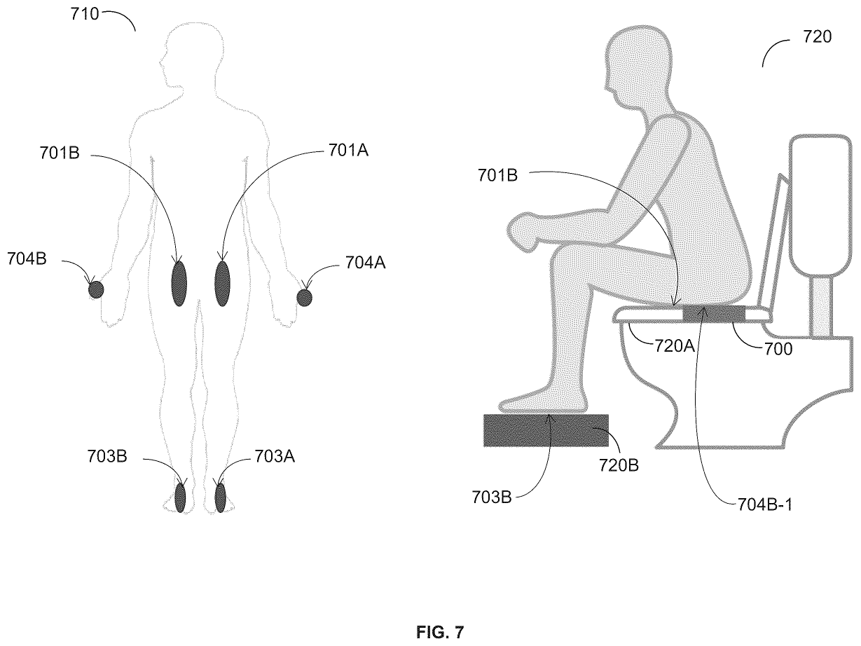

FIG. 7 is an example schematic diagram illustrating contact areas of a user that touch the MBD according to one embodiment. A human body diagram 710 includes pairs of contact areas, for example, but not limited to, buttocks 701A and 701B, soles of the feet 703A and 703B, and fingers 704A and 704B. In the example human body diagram 710, the contact areas are shown in pairs, indicated as A and B, where A areas are on one side of the body and B areas are on the other side of the body.

An in-use schematic diagram 720 illustrates a side view of the user in operation of the MBD. The contact areas of one side of the human body are shown to come in contact with different components of the MBD, the toilet seat 720A, the base pad footrest 720B, and the toilet seat controls 700. In the example embodiment, the buttock 701B contacts the toilet seat 720A, the sole of the foot 703B contacts the base pad footrest 720B, and the fingers may come in contact at point 704B-1 the toilet seat controls 700. It should be noted that, during operation of the MBD, the contact areas 701A through 704B of the human body contacts the plurality of sensors included in the components of the MBD and thus, the plurality of sensors can collect data.

According to the disclosed embodiments, data collection via the MBD may be performed automatically without always requiring a user to apply or activate the sensors intentionally. In order to accommodate multifunctional aspects of the MBD and optimize the available space, some signal processing functions may be performed outside of the device by transmitting the data signal from the device over to a user device and a cloud-based platform (e.g., user device 140; evaluation system 130, FIG. 1A-1B) for advanced data processing, evaluation, and interpretation. It should be noted that processing outside of the MBD conserves resources at the MBD by reducing memory and computing power for improved computing and device efficiency. It should be further noted the MBD complexity and size may be reduced through transmission of data signals and processing external to the MBD.

In an embodiment, the evaluation system (e.g., the evaluation system 130, FIG. 1A-1B) is configured to perform one or more evaluations such as, but not limited to, body weight, smarty body scale, fecal evaluation, urine evaluation, oxygen saturation, electrocardiogram, ambient conditions monitoring, fingerprint scanning, GPS, biometric identification, and the like. In a further embodiment, the evaluation system and/or the user device is configured to analyze data to perform communications, input device management, output management, speaker and/or microphone communications, and the like. It should be noted that the evaluation methods for certain types of evaluation discussed herein below are for illustrative purposes and do not limit the scope of the disclosed embodiments.

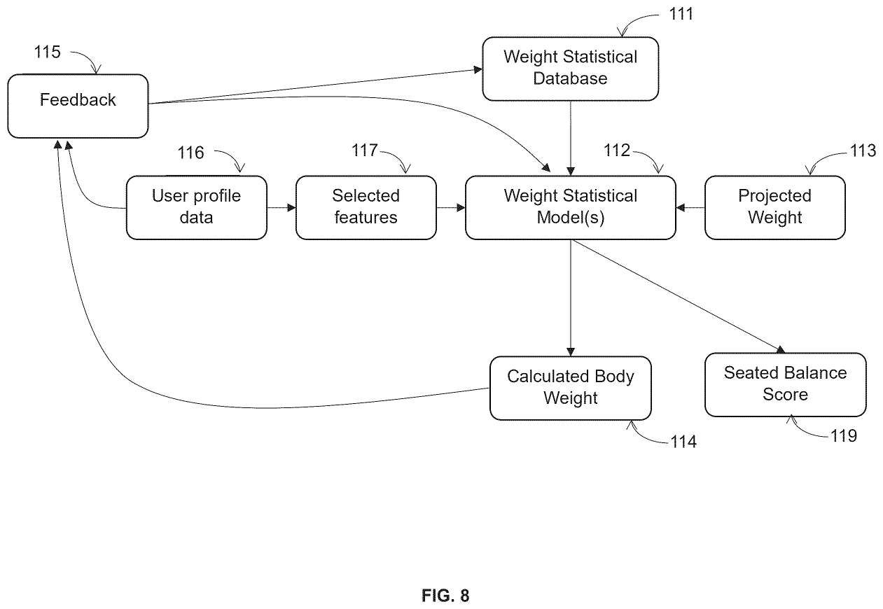

FIG. 8 is an example flow diagram illustrating a method of body weight evaluation according to an embodiment. The MBD is utilized to collect body weight data and the method of evaluation described herein may be executed by the evaluation system 130, FIG. 1A.

On a conventional weigh scale or a bathroom scale, a user is able to step on a platform and is able to position the entire weight to measure their Body Weight. In case of MBD, when a user is seated on the toilet seat, the entire body weight is not projected. In order to address this scenario, and measure the entire body weight, the disclosed embodiments leverage following inputs, without limitations, projected weight captured by contact points of the MBD (e.g., contact points 2A, 2B, 2C, 2D, FIG. 4-6) at the time of use, user profile data, and the like. As described in the previous figures, each contact point is associated to a pressure transducer that converts projected weight at each point into a corresponding electrical signal for evaluation. In an embodiment, the user profile data includes, for example, but not limited to, user entered body weight (at initial set-up or updated thereafter), user entered height, body mass index value (evaluated and recorded each time user uses MBD), gender, and the like, and any combination thereof.

A weight statistical database 111 includes initial set of manually curated readings from different users. These readings include, for example, body weight, projected body weight at each of the contact points for selected users in different seating patterns on the toilet seat, as well as additional user features (e.g., height, BMI, gender, etc.). In an embodiment, the weight statistical database may append additional entries into the database by gathering feedback 115 from each of the plurality of users using the toilet seat of the MBD (e.g., the toilet seat 120A, FIG. 1B). Data from the weight statistical database 111 is utilized by at least one weight statistical model 112.

At least one user specific feature 117 such as, but not limited to, actual reported weight, height, BMI, gender, and the like, are extracted from a user profile data 116. It should be noted that every user may not have maintained all fields in the user profile data 116, so this step will pull any of the available known user specific features 117 and pass on to the at least one weight statistical mode 112 for actual weight prediction.

The at least one weight statistical model 112 is an advanced statistical machine learning model that may be, without limitation, a supervised model, an unsupervised model, or a combined machine learning model. The model 112 is initially trained based on the initially curated entries in the weight statistical database 111. With usage, the at least one weight statistical model 112 further gathers feedback 115 to be continually trained. At the time of every usage, the at least one weight statistical model 112 picks up multiple readings from a projected weight 113 at individual contact points on the MBD and user specific features 117 to predict a calculated body weight 114 for the user. In an embodiment, the calculated body weight 114 is time-stamped and stored with additional user measurements collected during the usage along with the ambient condition measurements (e.g., temperature, humidity, elevation, etc.), and the location of the MBD. It should be noted that the MBD could be installed in a mobile home or a user may be using multiple toilet seats in a home, so the location and ambient conditions provide any noteworthy ambient conditions and user behavior for health risk analysis.

In a further embodiment, a seated balance score 119 is determined based on changes in reading at each of the contact points (e.g., 2A, 2B, 2C, and 2D, FIG. 6) during usage of the toilet seat of the MBD. In an example embodiment, the seated balance score 119 together with user profile data 116, for example, demographics, usage patterns, and more, may be an indicator of neurological or musculoskeletal disorders.

It should be noted that, in the method of body weight evaluation described herein using the MBD, there is no active user engagement required to measure the weight. As the user uses the bathroom deployed with the MBD, validated readings are automatically saved in the cloud and the user and their trusted team members may be alerted only when there is a persistent change over a scientifically determined period of usage. The reading and collection of user data with the MBD eliminates unnecessary anxiety of measuring every day and experiencing emotional highs and lows by a user based on manual reading.

Additionally, the disclosed embodiments of performing body weight evaluation are advantageous over traditional bathroom scales to address their limitations. As an example, when a scale is moved, traditional scales need to be calibrated to reset the internal parts and ensure accurate readings. However, given the fixed degree motion of the attacked MBD to the bathroom seat, the MBD can be easily auto calibrated before use. As another example, unlike traditional scales that require a hard leveled surface and is less accurate on rugs, the MBD is always on a stable bathroom toilet base reducing inaccuracies from misplacement. As yet another example, traditional bathroom scales often require a user to stand still for few seconds for precise results, which can be hard to enforce. However, with the disclosed MBD, average users will be on the seat for 20 seconds on average, providing ample time for readings collection.

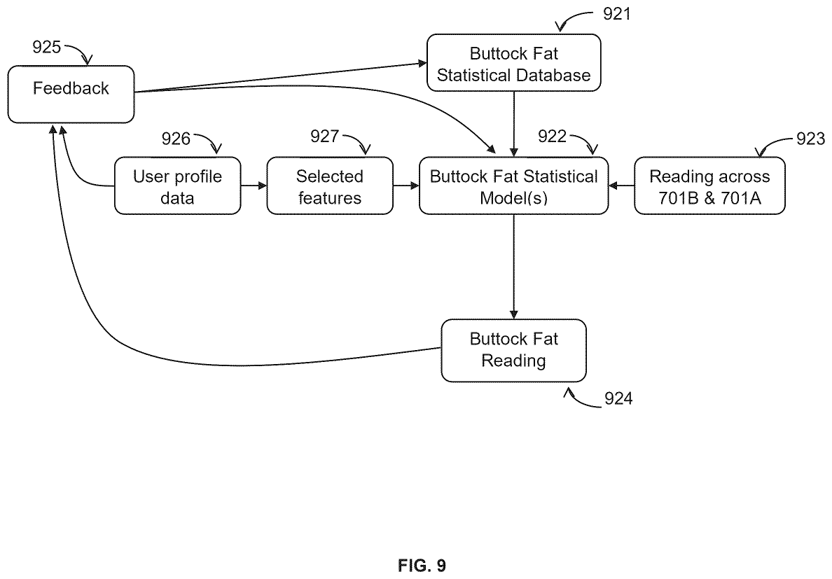

Evaluation of fat distribution and content in the human body is highly desired to monitor well-being of users. The human body stores fat in multiple places. A layer of fat that wraps around the inner organs, and is typically associated with a large waist or belly, is called as visceral fat and drives the risk for disease, such as, diabetes, heart disease, stroke, and even dementia. Other remaining non-visceral fat are found just below the skin and is not related to many of the classic obesity-related risks, and some evidence even suggests it might even be protective. Such non-visceral fat is stored in, for example, but not limited to buttocks, thighs, and the like. The distribution of visceral and non-visceral fat is influenced by, for example, environment, lifestyle, demographics variables such as ethnicity, gender, age, and other variables such as pregnancy and menopause (in women). Therefore, methods to effectively determine visceral and non-visceral fat distributions are desired to identify early health risks and are discussed herein below in FIGS. 9-11. It should be noted that the disclosed embodiments allow collection of multiple measurements passively, without active user intervention, in evaluation of visceral and non-visceral fat.

FIG. 9 is an example flow diagram illustrating a method of buttock fat evaluation according to an embodiment. The method of evaluation described herein may be executed by the evaluation system 130, FIG. 1A.

In an embodiment, bioimpedance measurements are collected from a user who is seated at the toilet seat of the MBD 420A, FIG. 4. The toilet seat 420A sends bioimpedance signals across the buttock region (e.g., 701A and 701B, FIG. 7) across the sensors of the toilet seat (e.g., 1A and 1F of 420A, FIG. 4). In another embodiment, the bioimpedance measurements are collected from a user in contact with bioimpedance electrodes at multiple body parts. The bioimpedance electrodes may be free-form electrodes connected to the MBD. In an example embodiment, the bioimpedance electrodes are connected to a user's wrist and ankle for the bioimpedance measurement. In some embodiments, the bioimpedance measurements may be collected from other parts of the body, for example, arm, abdominal, neck, and the like, and more, based on a configuration of the MBD may include electrodes in different locations of the body. It should be noted that the method is described with respect to buttocks fat for illustrative purposes and does not limit the scope of the various disclosed embodiments.

A buttock fat statistical database 921 includes an initial set of manually curated readings from different users. These readings include selected user features 927 such as, for example, body weight, corresponding reading across buttocks contact areas (e.g., 701B and 701A, FIG. 7) 923 and corresponding user profile data 926 such as, but not limited to, height, BMI, age, gender, ethnicity, other known conditions (e.g., pregnancy, diabetes etc.), and more. The buttock fat statistical database 921 gather feedback 925 and may append additional entries into the database 921 upon ongoing use of the MBD by one or more users.

At least one buttock fat statistical model 922 is configured to receive data from at least one of the buttock fat statistical database 921, reading across buttock contact areas 923, feedback 925, and selected user features 927 to determine buttock fat reading 924. The at least one buttock fat statistical model 922 is initially trained based on manually curated entries in the buttock fat statistical database 921. In an embodiment, the buttock fat statistical model 922 may apply a machine learning algorithm, such as, supervised, unsupervised, or a combination of supervised and unsupervised machine learning algorithms to be further trained using feedback 924 gathered with usage. In an embodiment, at time of usage, the at least one buttock fat statistical model 922 picks up multiple readings which are evaluated for the buttock fat reading 924. In a further embodiment, the determined buttock fat reading 924 is time-stamped and stored, for example, at the buttock fat statistical database 921.

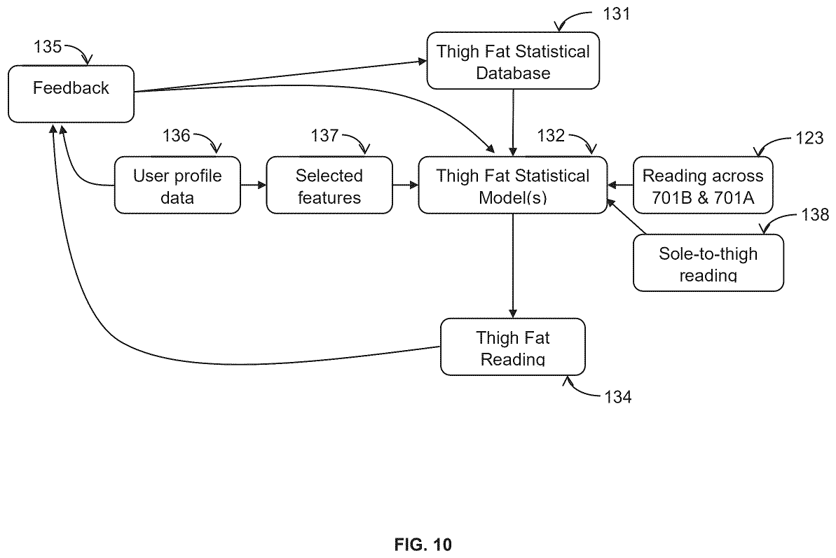

FIG. 10 is an example flow diagram illustrating a method of thigh fat evaluation according to an embodiment. The method of evaluation described herein may be executed by the evaluation system 130, FIG. 1A.

In an embodiment, bioimpedance measurements are collected from a user seated at the toilet seat with legs rested on the base pad footrest of the MBD 420A and 420B, FIG. 4. The MBD is configured to collect bioimpedance signals across the sole to the buttock of the same side for each side. As an example, bioimpedance signals are read from the left buttock to the left sole (e.g., 701B and 703B, FIG. 7) using the left-side sensors of the MBD (e.g., 1F and 3B, FIG. 4) and from the right buttock to the right sole (e.g., 701A and 703A, FIG. 7) using the right-side sensors of the MBD (e.g., 1A and 3A, FIG. 4). In another embodiment, the bioimpedance measurements are collected from a user in contact with bioimpedance electrodes at multiple body parts. The bioimpedance electrodes may be free-form electrodes connected to the MBD.

A thigh fat statistical database 131 includes initial set of manually curated readings from different users. These readings include selected user features such as, but not limited to, body weight, corresponding readings across the left buttock to the left sole (e.g., 701B and 703B, FIG. 7), the right buttock to the right sole (e.g., 701A and 703A, FIG. 7), and corresponding buttock fat reading at buttock contact areas (e.g., 701B and 701A, FIG. 7) 123 as well as selected features 137 such as, but not limited to, height, BMI, gender, ethnicity, other conditions (e.g., pregnancy, diabetes, etc.) that are extracted from a user profile data 136. In an embodiment, the thigh fat statistical database 131 may append additional entries into the database by gathering feedback 135 from each of the plurality of users using the MBD.

At least one thigh fat statistical model 132 is configured to receive data from the thigh fat statistical database 131, reading across buttock contact areas 123, sole-to-thigh readings for each side of the body 138, feedback 135, and selected user features 137 to determine thigh fat reading 134. The at least one thigh fat statistical model 132 is initially trained based on manually curated entries in the thigh fat statistical database 131. In an embodiment, the thigh fat statistical model 132 may apply a machine learning algorithm, such as, supervised, unsupervised, or a combination of supervised and unsupervised machine learning algorithms to be further trained using feedback 135 gathered with usage. In an embodiment, at time of usage, the at least one thigh fat statistical model 132 picks up multiple readings which are evaluated for the thigh fat reading 134. In a further embodiment, the determined thigh fat reading 134 is time-stamped and stored.

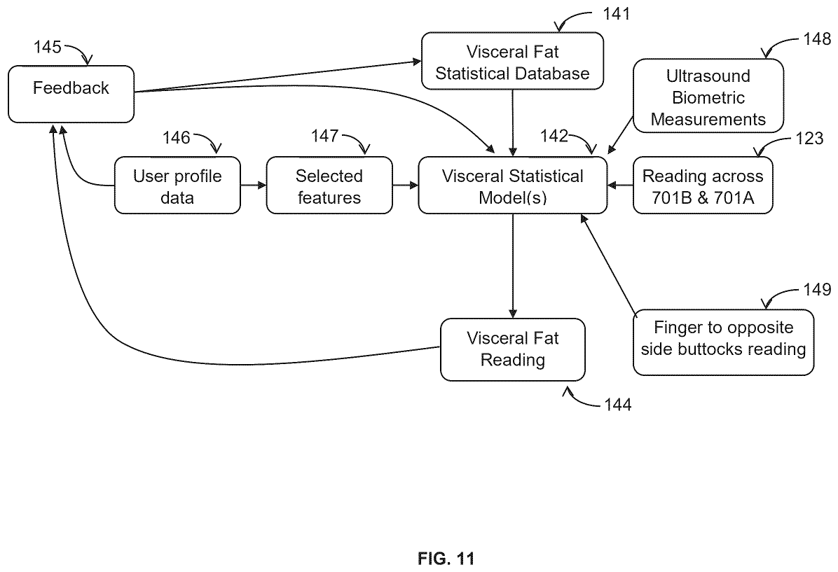

FIG. 11 is an example flow diagram illustrating a method of visceral fat evaluation according to an embodiment. The method of evaluation described herein may be executed by the evaluation system 130, FIG. 1A.

In an embodiment, bioimpedance measurements are collected from a user seated at the toilet seat and presses the toilet seat control of the MBD 420A and 200, FIG. 4. The MBD is configured to collect and send bioimpedance signals across a finger of one side of the body and a buttock of the other side of the body, for both sides. As an example, bioimpedance signals are read from the right hand finger to the left buttock (e.g., 704A and 701B, FIG. 7) and from the left hand finger to the right buttock (e.g., 704B and 701A, FIG. 7), which are utilized to determine the visceral fat readings. It should be noted that the disclosed embodiments are described with respect to measurements from the fingers and buttocks for illustrative purposes and does not limit the scope of the disclosed embodiments. In an example embodiment, readings may be collected from other parts of the body such as, but not limited to, abdominal area, arm, neck, and the like, and more.

In another embodiment, the bioimpedance measurements are collected from a user in contact with bioimpedance electrodes at multiple body parts. The bioimpedance electrodes may be free-form electrodes connected to the MBD. In an example embodiment, the bioimpedance electrodes are connected to a user's wrist and ankle for the bioimpedance measurement.

In some embodiments, for example in pregnant women, a fetal mass (or weight) is accounted for in the visceral fat evaluation. In an example embodiment, the fetal weight is determined based on ultrasound biometric measurements such as a crown-rump length (CRL) that measures the distance between a top of the embryo and its rump, a biparietal diameter (BPD) between the two sides of the head that is measured after 13 weeks, a head circumference (HC) of the fetus' head that is measured after 13 weeks, and a femur length (FL) of the longest bone in the body and reflects the longitudinal growth of the fetus.

A visceral fat statistical database 141 includes initial set of curated readings from different users. These readings include selected user features such as, but not limited to, readings across the right hand finger to the left buttock contact area (e.g., 704A and 701B, FIG. 7) and across the left hand finger to the right buttock contact area (e.g., 704B and 701A, FIG. 7) 149, fetal ultrasound biometric information 148, and corresponding buttock fat reading at buttock contact areas (e.g., 701B and 701A, FIG. 7) 123 as well as selected features 147 such as, but not limited to, height, BMI, gender, ethnicity, other conditions (e.g., pregnancy, diabetes, etc.) that are extracted from a user profile data 146. The visceral fat statistical database 141 further includes data for pregnancy cases, including the ultrasound biometric information of the fetus. In an embodiment, the visceral fat statistical database 141 may append additional entries into the database by gathering feedback 145 from each of the plurality of users using the MBD.

At least one visceral fat statistical model 142 is configured to receive data from at least one of the visceral fat statistical database 141, reading across buttock contact areas 123, finger to opposite side buttock readings 149, feedback 145, ultrasound biometric measurements 148, and selected user features 147 to determine visceral fat reading 134. In an embodiment, the final weight including visceral fat weight, of a user is adjusted by determined fetal weights assessed based on the ultrasonic biometric measurements 148. The at least one visceral fat statistical model 142 is machine learning model that is initial trained based on initially curated entries in the visceral fat statistical database 141. The visceral fat statistical model 132 may apply a machine learning algorithm, such as, supervised, unsupervised, or a combination of supervised and unsupervised machine learning algorithms and is continually trained by gathering feedback 145. In an embodiment, at time of usage, the at least one visceral fat statistical model 142 picks up multiple readings which are evaluated for the visceral fat reading 144. In a further embodiment, the determined visceral fat reading 144 is time-stamped and stored.

The method described to determine visceral fat evaluation in FIG. 11 may be applied to determine skeletal muscle mass. In such a scenario, the skeletal muscle mass model, skeletal muscle mass database, and skeletal muscle mass reading is employed at the steps of 141, 142, and 144.

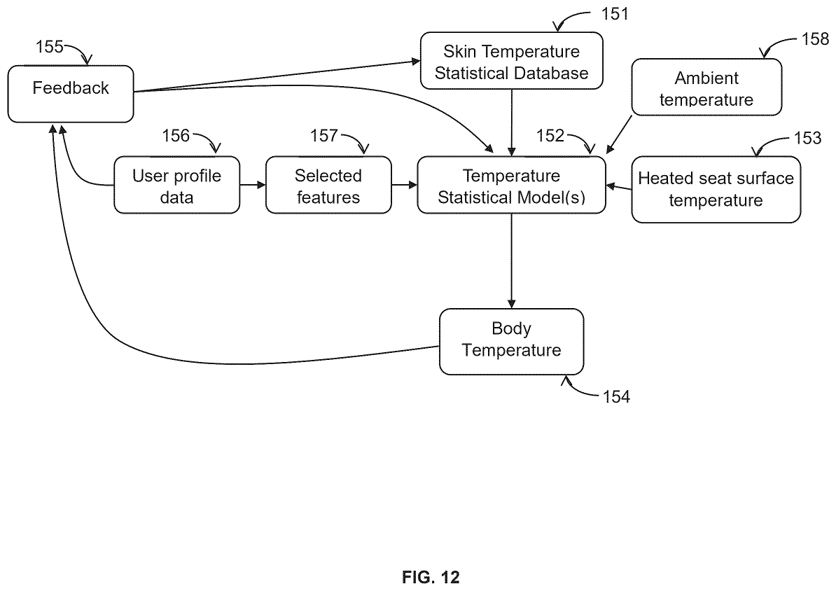

FIG. 12 is an example flow diagram illustrating a method of body temperature evaluation according to an embodiment. The method of evaluation described herein may be executed by the evaluation system 130, FIG. 1A based on data collected by the MBD.

Measuring a body temperature is very important for tracking a wellbeing of a person. A number of diseases are characterized by a change in body temperature or can be tracked by measuring body temperature. Additionally, individuals with limited (temporary or permanent) ability due to age (e.g., very young or older), lifestyle (e.g., alcohol or drug intake), medical condition (e.g., Parkinson's disease, anorexia nervosa, hypothyroidism, severe form of arthritis, stroke, etc.) can significantly benefit from the passive monitoring of the body temperature.

Body temperature is driven primarily by the amount of heat a body generates versus the amount of heat the body is able to dissipate. Under resting conditions, heat production within the body is the primary driver of the body temperature and is driven mainly by the metabolic activity of inner organs such as liver, intestines, kidneys, heart in the abdominal/thoracic cavity, and the brain, together producing approximately 70% of the entire resting metabolic rate of the human body. However, this core internal heat is generated in only 8% of the body mass with a surrounding skin surface of only about 0.3 meter-square, compared to overall surface area of an average person (e.g., 5 feet 9 inches, 155 lbs) that is about 1.8 meter-square, thus creating challenges to detect body temperature at body surfaces.

Moreover, proximal skin surface is not ideally shaped, for example, too flat, and does not effectively transfer heat to the environment. Heat exchange with the environment occurs by means of conduction, convection, radiation, and evaporation (enabled by perspiration). In addition, the body temperature can largely vary depending on various factor such as, but not limited to, activities undertaken, course of day (e.g., morning, evening, etc.), environmental conditions (e.g., temperature, air movement, humidity, sunshine, microclimate under clothing, bedding, etc.), demographics (e.g., age, gender, medical conditions, etc.), food or drink consumed, ovulation in women, and more. Thus, isolation of these various factors to determine a true baseline for a person is challenging. As a result, many complexities exist in effectively measuring the body temperature from an external bodily surface.

To this end, the disclosed embodiments provide a method to continuously measure body surface temperature by leveraging one or more surface temperature sensors on the components of the MBD (e.g., toilet seat 420A, base pad footrest 420B, toilet seat control 220, FIG. 4). The temperature sensed by the surface temperature sensors includes temperature inside the user's body and ambient factors (e.g., temperature, humidity, etc.) that are collected during the day without imposing inconveniences onto the user and are adjusted using the algorithm discussed herein. It should be noted that the standardization of time and body area are critical for on-going body temperature data collection and evaluation.

In an embodiment, surface temperatures are collected from a user seated at the toilet seat of the MBD 420A, FIG. 4 and, in some embodiments, also the user with their foot resting on the base pad footrest 420B, FIG. 4. In a further embodiment, ambient temperatures of a user surrounding are collected using temperatures sensors deployed at the MBD (e.g., 420A, 420B, and 200, FIG. 4). It should be noted that the disclosed embodiments are described with respect to measurements from the toilet seat and the footrest for illustrative purposes and does not limit the scope of the disclosed embodiments. Some example configurations of collecting surface temperature may include, without limitation, free-form temperature sensor, wearable devices, and the like, and more.

A skin temperature statistical database 151 includes an initial set of curated readings from different users. These readings include selected user features such as, but not limited to, body weight, corresponding readings across at least one contact area (e.g., buttocks 701A, 701B and foot soles 703A, 703B, FIG. 7) as well as selected features 157 such as, but not limited to, height, BMI, gender, ethnicity, other conditions (e.g., pregnancy, diabetes, etc.) that are extracted from a user profile data 156. In an embodiment, the temperature statistical database 151 may append additional entries into the database by gathering feedback 155 from each of the plurality of users using the MBD.

At least one temperature statistical model 152 is configured to receive data from the skin temperature statistical database 151, reading across at least one contact areas 153, ambient temperature reading 158, feedback 155, and selected user features 157 to determine body temperature reading 154. The at least one temperature statistical model 152 is initially trained based on manually curated entries in the skin temperature statistical database 151. In an embodiment, the temperature statistical model 152 may apply a machine learning algorithm, such as, supervised, unsupervised, or a combination of supervised and unsupervised machine learning algorithms to be further trained using feedback 155 gathered with usage. In an embodiment, at time of usage, the at least one temperature statistical model 152 picks up multiple readings which are evaluated for the body temperature reading 154. In a further embodiment, the determined body temperature reading 154 is time-stamped and stored.

It has been identified that an average person spends about 2 minutes per toilet visit and makes about six to seven visits per day. To this end, it should be noted that body temperature evaluation according to the disclosed embodiments enables daily physiological monitoring for temperature reading variations by collecting at least a couple of readings in a day, and makes the process of data collection entirely effortless for the user.

In women, body temperature increases by 0.72 °F (0.4 °C) at the time of ovulation. For women planning pregnancy, tracking body temperature daily can help understand the individual body patterns and such information could be used to plan intercourse for pregnancy. In case of women not planning for pregnancy, understanding of body temperature patterns can also be used to identify the days to avoid intercourse.

The average normal human body temperature is around 98.6 °F or 37 °C, however, based on research that “normal” can be anywhere from 97 °F (36 °C) to 99 °F (37.2 °C). Temperature higher than 100.4 °F (38 °C) is considered fever, and the body temperature below 95 °F (35 °C) is considered hypothermia.

Fever is an important part of the body's defense against infection. Most bacteria and viruses that cause infections in people thrive best at 98.6 °F (37 °C). Many infants and children develop high fevers with mild viral illnesses. Almost any infection can cause a fever, including, but not limited to, bone infections (osteomyelitis), appendicitis, skin infections, meningitis, respiratory infections (e.g., colds or flu-like illnesses, sore throats, ear infections, sinus infections, mononucleosis, bronchitis, pneumonia), urinary tract infections, viral gastroenteritis and bacterial gastroenteritis, and the like. In addition, low-grade fever for 1 or 2 days is detected after some immunizations. Moreover, autoimmune or inflammatory disorders may also cause fevers.

Hypothermia has a spectrum from mild to severe. Temperatures can vary, but mild hypothermia is typically considered a body temperature between 89 °F-95 °F; moderate hypothermia is a body temperature between 82 °F-89 °F; and severe hypothermia is a body temperature lower than 82 °F. Hypothermia typically comes on gradually and gets progressively worse as it is left untreated. Hypothermia symptoms can commonly progress from, for example, but not limited to, cold feet, hands, and face all the way to loss of heartbeat, leading to death. Hypothermia may be caused by many different factors such as, but not limited to, alcohol or drug use, anesthesia, anorexia, hypothyroidism, malnutrition, medicines (e.g., antidepressants, antipsychotics, sedatives, etc.), nerve damage, Parkinson's disease, sepsis, stroke, Wilson's syndrome (or Wilson's temperature syndrome) which could be an indication of abnormal thyroid activity, and more.

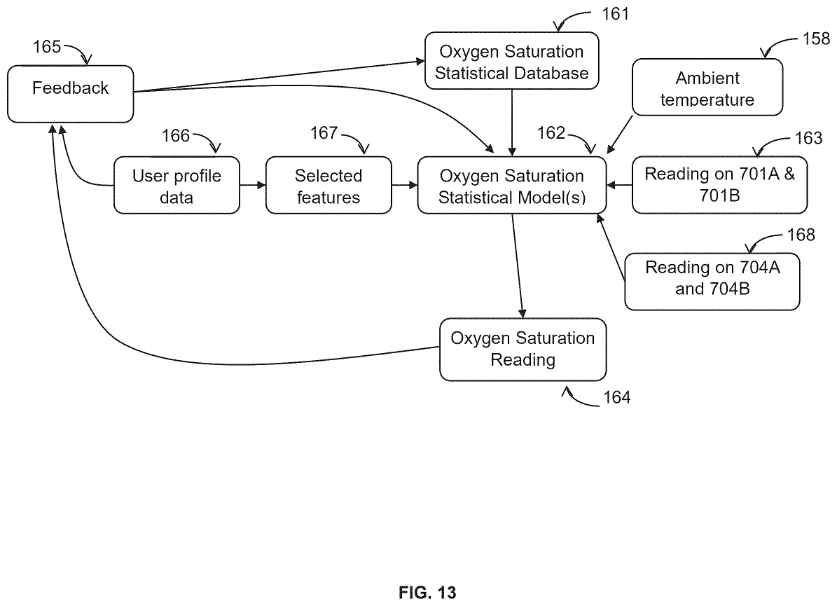

FIG. 13 is an example flow diagram illustrating a method of oxygen saturation evaluation according to an embodiment. The method of evaluation described herein may be executed by the evaluation system 130, FIG. 1A based on data (or reading) collected from users of the MBD.

Pulse oximetry is noninvasive method of indirectly determining blood oxygen saturation (SpO2) that is accepted as a reliable technique to assess respiratory function. A difference in light absorption of oxygenated and deoxygenated hemoglobin are measured to calculate the ratio of oxyhemoglobin (HbO2) to the total concentration of hemoglobin in blood. Such conventional technique is used to check for a person's health conditions such as, but not limited to, heart condition, lung and respiratory condition, blood hemoglobin levels, oxygen deprivation (e.g., difficulty breathing, shortness of breath, dizziness, etc.), and the like, which affect blood oxygen levels.

The traditional pulse oximetry sensors are placed on the head, face, hands, or feed as a result of strong blood perfusion, accessibility, and lower inter-subject variability at these sites. However, it has been identified that measurements from other body sites such as the torso, arms, thighs, and legs have higher inter-subject variability as influence from varying blood perfusion factors as well as physiologic characteristics such as adipose tissue, which accumulate disproportionally on relevant body sites, the torso, legs, and upper arms. The method described herein, according to the disclosed embodiments, provides a non-invasive technique to determine blood oxygen saturations from such body sites identified to have higher inter-subject variability. It should be noted that oxygen saturation evaluation from body parts, such as, thighs, arms, legs, and the like, using the MBD enables automatic collection and continual tracking of a user's oxygen saturation levels for monitoring user's health conditions without requiring active user engagement.

In an embodiment, oxygen saturations are determined based on backscatter of light measured from a user in operation of the MBD, seated at the toilet seat 420A with feet and fingers resting on the base pad footrest 420B and toilet seat control 200, of FIG. 4, respectively. The measurements of oxygen saturation leverages reflectance type pulse oximetry that uses light detector sensors located adjacent to emitter sensors to measure the backscatter of light from various body sites. In an embodiment, light detector sensors may be located at the toilet seat, base pad footrest, and the toilet seat control of the MBD to come in contact with contact areas of the body (e.g., 701A and 702B, 703A and 703B, and 704A and 704B, respectively, FIG. 7). It should be noted that other contact areas of the body may be utilized without departing the scope of the disclosed embodiments. It should be further noted that the toilet sear configuration of the MBD is for illustrative purposes and does not limit the scope of the disclosed embodiments. The oxygen saturation signals may be collected from sensors in other formats.

An oxygen saturation statistical database 161 includes an initial set of curated readings from different users. These readings include selected user features such as, but not limited to, body weight, corresponding readings across at least two contact area (e.g., buttocks 701A, 701B, foot soles 703A, 703B, finger 704A, 704B FIG. 7) as well as selected features 167 such as, but not limited to, height, BMI, gender, ethnicity, other conditions (e.g., pregnancy, diabetes, etc.) that are extracted from a user profile data 166. In an embodiment, the oxygen saturation statistical database 161 may append additional entries into the database by gathering feedback 165 from each of the plurality of users using the MBD.

At least one oxygen saturation statistical model 162 is configured to receive data from the oxygen saturation statistical database 161, reading surface light reflection across buttock contact areas (e.g., 701A and 701B, FIG. 7) 163, reading surface light reflection across finger (e.g., 704A and 704B, FIG. 7) 168, ambient temperature reading 158, feedback 165, and selected user features 167 to determine blood oxygen saturation reading 164. The at least one oxygen saturation statistical model 162 is initially trained based on manually curated entries in the oxygen saturation statistical database 161. In an embodiment, the oxygen saturation statistical model 162 may apply a machine learning algorithm, such as, supervised, unsupervised, or a combination of supervised and unsupervised machine learning algorithms to be further trained using feedback 165 gathered with usage. In an embodiment, at time of usage, the at least one oxygen saturation statistical model 162 picks up multiple readings which are evaluated for the blood oxygen saturation reading 164. In a further embodiment, the determined blood oxygen saturation reading 164 is time-stamped and stored.

According to the disclosed embodiments, oxygen saturation levels are determined by measuring and determining intra-individual ratios of perfusion between body regions. Although individuals may have varying degrees of absolute perfusion, when considering the same body regions, the relative intra-individual ratios of perfusion between the body regions are similar between the individuals. To this end, intra-individual relative imaging is utilized to normalize perfusion data to minimize variations that occur from use of absolute LDI perfusion imaging in conventional methods. Such intra-individual relative imaging for determining oxygen saturation overcomes deficiencies in conventional pulse oximetry techniques that includes motion artifacts, low-pulse amplitude, response delay, and more that reduce accuracy.

It should be appreciated that the disclosed embodiments provide a solution to collect multiple blood oxygen level measures of a user over time and use modern advanced statistical methods to assess the baseline for the user profile category and identify noteworthy deviations from the established baseline. The statistical engine has a learning ability to review changes in the user profile and use that information to adjust the user profile category and subsequently reevaluate baseline on an ongoing basis. It should be further appreciated that even for the purpose of measurements itself, the disclosed embodiments have the benefit of making corrections based on other measurements collected therewithin.

As noted above, the MBD and evaluation system is utilized to measure and evaluate various functionalities related to a user and the user environment. In an example embodiment, urinalysis (or urine evaluation) is performed for urine-based tests, for example, but not limited to, urine color evaluation, ovulation, pregnancy, urinary tract infection, yeast infection, substance use, other human condition monitoring, and more. In another example embodiment, fecal evaluation is used to check stool samples for visual information and variety of substances including blood. In another embodiment, a photoplethysmogram (PPG) analysis is performed to measure fluctuations in the blood volume which are caused by the mechanical pressure pulses.

In an example embodiment, the MBD is configured to measure and determine satellite navigation systems that uses satellites to provide autonomous geo-spatial positioning. Small electronic receivers are utilized to determine their location (e.g., longitude, latitude, altitude, elevation, etc.) using time signals transmitted along a line of sight by radio from satellites. The signals also allow the electronic receiver to calculate the current local time to high precision, which allows time synchronization. In another example embodiment, the MBD is configured to perform fingerprint scanning to capture the fingerprints that are a unique marker for a person for identification of users. The fingerprints are defined by the impression of the friction ridges of a human finger that are unique for individuals. In yet another example embodiment, the MBD may be communicating connected to a mobile application. also referred to as a mobile app or simply an app, of, for example, a user device (e.g., mobile phone, tablet, smart watch, etc.). The mobile application is a computer program of software application for variety of functions associated with the MBD and/or evaluation system, including remote controlling of MBD, data analytics as well as interactions with the MBD.

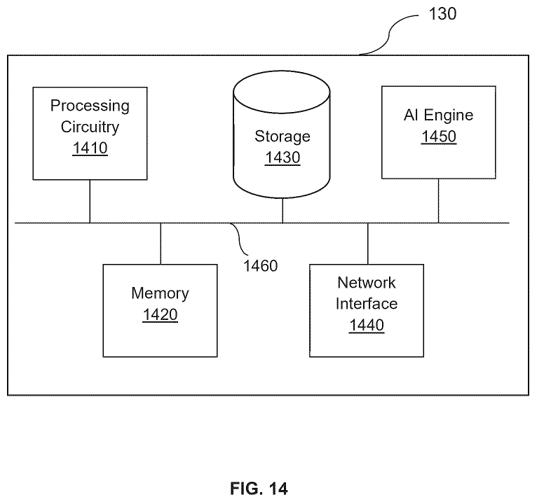

FIG. 14 is an example schematic diagram of an evaluation system 130 according to an embodiment. The evaluation system 130 includes a processing circuitry 1410 coupled to a memory 1420, a storage 1430, a network interface 1440, and an artificial intelligence (AI) engine 1450. In an embodiment, the components of the evaluation system 130 may be communicatively connected via a bus 1460.

The processing circuitry 1410 may be realized as one or more hardware logic components and circuits. For example, and without limitation, illustrative types of hardware logic components that can be used include field programmable gate arrays (FPGAs), application-specific integrated circuits (ASICs), Application-specific standard products (ASSPs), system-on-a-chip systems (SOCs), graphics processing units (GPUs), tensor processing units (TPUs), general-purpose microprocessors, microcontrollers, digital signal processors (DSPs), and the like, or any other hardware logic components that can perform calculations or other manipulations of information.

The memory 1420 may be volatile (e.g., random access memory, etc.), non-volatile (e.g., read only memory, flash memory, etc.), or a combination thereof.

In one configuration, software for implementing one or more embodiments disclosed herein may be stored in the storage 1430. In another configuration, the memory 1420 is configured to store such software. Software shall be construed broadly to mean any type of instructions, whether referred to as software, firmware, middleware, microcode, hardware description language, or otherwise. Instructions may include code (e.g., in source code format, binary code format, executable code format, or any other suitable format of code). The instructions, when executed by the processing circuitry 1410, cause the processing circuitry 1410 to perform the various processes described herein.

The storage 1430 may be magnetic storage, optical storage, and the like, and may be realized, for example, as flash memory or other memory technology, compact disk-read only memory (CD-ROM), Digital Versatile Disks (DVDs), or any other medium which can be used to store the desired information.

The network interface 1440 allows the evaluation system 130 to communicate with, for example, the network 110.

The AI engine 1450 may be realized as one or more hardware logic components and circuits, including graphics processing units (GPUs), tensor processing units (TPUs), neural processing units, vision processing units (VPU), reconfigurable field-programmable gate arrays (FPGA), and the like. The AI engine 1450 is configured to perform, for example, machine learning based on input data such as user profile data, readings from one or more sensors on the MBD (e.g., of user and/or ambient conditions), feedback data collected from MBD, selected user features, and more, received over the network 110.

It should be understood that the embodiments described herein are not limited to the specific architecture illustrated in FIG. 14, and other architectures may be equally used without departing from the scope of the disclosed embodiments.

The various embodiments disclosed herein can be implemented as hardware, firmware, software, or any combination thereof. Moreover, the software is preferably implemented as an application program tangibly embodied on a program storage unit or computer readable medium consisting of parts, or of certain devices and/or a combination of devices. The application program may be uploaded to, and executed by, a machine comprising any suitable architecture. Preferably, the machine is implemented on a computer platform having hardware such as one or more central processing units (“CPUs”), a memory, and input/output interfaces. The computer platform may also include an operating system and microinstruction code. The various processes and functions described herein may be either part of the microinstruction code or part of the application program, or any combination thereof, which may be executed by a CPU, whether or not such a computer or processor is explicitly shown. In addition, various other peripheral units may be connected to the computer platform such as an additional data storage unit and a printing unit. Furthermore, a non-transitory computer readable medium is any computer readable medium except for a transitory propagating signal.

All examples and conditional language recited herein are intended for pedagogical purposes to aid the reader in understanding the principles of the disclosed embodiment and the concepts contributed by the inventor to furthering the art, and are to be construed as being without limitation to such specifically recited examples and conditions. Moreover, all statements herein reciting principles, aspects, and embodiments of the disclosed embodiments, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

It should be understood that any reference to an element herein using a designation such as “first,” “second,” and so forth does not generally limit the quantity or order of those elements. Rather, these designations are generally used herein as a convenient method of distinguishing between two or more elements or instances of an element. Thus, a reference to first and second elements does not mean that only two elements may be employed there or that the first element must precede the second element in some manner. Also, unless stated otherwise, a set of elements comprises one or more elements.

As used herein, the phrase “at least one of” followed by a listing of items means that any of the listed items can be utilized individually, or any combination of two or more of the listed items can be utilized. For example, if a system is described as including “at least one of A, B, and C,” the system can include A alone; B alone; C alone; 2A; 2B; 2C; 3A; A and B in combination; B and C in combination; A and C in combination; A, B, and C in combination; 2A and C in combination; A, 3B, and 2C in combination; and the like.

1. A method for simultaneously determining a plurality of health parameters, comprising:
collecting sensor data from a multifunctional body device (MBD), wherein the sensor data include impedance cardiography (ICG) sensor data and electrocardiogra (ECG) sensor data;
preprocessing the ICG sensor data and the ECG sensor data to remove artifacts, by applying a first machine learning algorithm, from each of the ICG sensor data and the ECG sensor data, wherein the artifacts have signal interferences between the ICG sensor data and the ECG sensor data;
extracting sensor features from each of the preprocessed ICG sensor data and the ECG sensor data, wherein the sensor features identify sensor data characteristics for each of the ICG sensor data and the ECG sensor data;
determining the plurality of health parameters by applying an evaluation model to the extracted sensor features from the ICG sensor data and the ECG sensor data, wherein the evaluation model is a multi-layer neural network model;
aggregating the determined plurality of health parameters to determine a trend of the plurality of health parameters, wherein the trend is monitored over time; and
generating a report on the determined trend of the plurality of health parameters, wherein the generated report is caused to be displayed to a user.

2. The method of claim 1, wherein the ICG sensor data and the ECG sensor data are collected simultaneously.

3. The method of claim 1, wherein the preprocessing of the ICG sensor data and the ECG sensor data further comprises:
generating a first derivative for each of the ICG sensor data and the ECG sensor data;
labeling the artifacts on the ICG sensor data and the ECG sensor data; and
generating a synchronized ICG-ECG preprocessed sensor data, wherein the synchronized ICG-ECG preprocessed sensor data aligns the preprocessed ICG sensor data and the preprocessed ECG sensor data at a single temporal scale.

4. The method of claim 1, wherein the artifacts are caused by at least one of: breathing, movement, pressure, and sensor interference.

5. The method of claim 1, wherein the sensor features include at least one of: waveform characteristics, derived parameters, time-domain features, and frequency-domain features.

6. The method of claim 1, wherein the collected sensor data includes a bioimpedance measurement, wherein the bioimpedance measurement is collected from the MBD at a different time than the ICG sensor data and ECG sensor data, wherein the plurality of health parameters from the bioimpedance measurement includes body composition parameters.

7. The method of claim 1, wherein the plurality of health parameters includes indicate cardiovascular health values.

8. (canceled)

9. A system for simultaneously determining a plurality of health parameters, comprising:
a processing circuitry; and
a memory, the memory containing instructions that, when executed by the processing circuitry, configure the system to:
collect sensor data from a multifunctional body device (MBD), wherein the sensor data include impedance cardiography (ICG) sensor data and electrocardiogra sensor data;
preprocess the ICG sensor data and the ECG sensor data to remove artifacts, by applying a first machine learning algorithm, from each of the ICG sensor data and the ECG sensor data, wherein the artifacts have signal interferences between the ICG sensor data and the ECG sensor data;
extract sensor features from each of the preprocessed ICG sensor data and the ECG sensor data, wherein the sensor features identify sensor data characteristics for each of the ICG sensor data and the ECG sensor data;
determine the plurality of health parameters by applying an evaluation model to the extracted sensor features from the ICG sensor data and the ECG sensor data, wherein the evaluation model is a multi-layer neural network model;
aggregate the determined plurality of health parameters to determine a trend of the plurality of health parameters, wherein the trend is monitored over time; and
generate a report on the determined trend of the plurality of health parameters, wherein the generated report is caused to be displayed to a user.

10. The system of claim 9, wherein the ICG sensor data and the ECG sensor data are collected simultaneously.

11. The system of claim 9, wherein the system is further configured to:
generate a first derivative for each of the ICG sensor data and the ECG sensor data;
label the artifacts on the ICG sensor data and the ECG sensor data; and
generate a synchronized ICG-ECG preprocessed sensor data, wherein the synchronized ICG-ECG preprocessed sensor data aligns the preprocessed ICG sensor data and the preprocessed ECG sensor data at a single temporal scale.

12. The system of claim 9, wherein the artifacts are caused by at least one of: breathing, movement, pressure, and sensor interference.

13. The system of claim 9, wherein the sensor features include at least one of: waveform characteristics, derived parameters, time-domain features, and frequency-domain features.

14. The system of claim 9, wherein the collected sensor data includes a bioimpedance measurement, wherein the bioimpedance measurement is collected from the MBD at a different time than the ICG sensor data and ECG sensor data, wherein the plurality of health parameters from the bioimpedance measurement includes body composition parameters.

15. The system of claim 9, wherein the plurality of health parameters includes cardiovascular health values.

16-24. (canceled)

25. A multifunctional body device (MBD), comprising:
an electrocardiogram that includes at least three ECG leads;
an impedance cardiography (ICG) system that includes at least two pairs of ICG electrodes;
a body composition system that includes at least one bioimpedance electrode;
a calibration system that is configured to calibrate raw sensor data collected via the at least three ECG leads and the at least two pairs of ICG electrodes, wherein the calibration system includes reference calibration resistors; and
a network interface for observing network traffic.

26. The device of claim 25, wherein the device further comprises:
a power management unit; and
a bus configured to allow communication among the ICG system, the ECG system, the body composition system, and the calibration system.

27. The device of claim 25, wherein at least the ICG system, the ECG, the body composition system, and the calibration system are integrated into a single system on chip (SoC).

28. The device of claim 25, wherein at least the ICG system, the ECG, the body composition system, and the calibration system are integrated into a printed circuit board.

29. The device of claim 25, wherein the at least one bioimpedance electrode and the at least two pairs of the ICG electrodes are separately at least one of:
gel electrodes, Transcutaneous electrical nerve stimulation (TENS) electrodes, and metal-based electrodes.

30. The device of claim 25, wherein the device further comprises:
a body weight scale;
a bidet;
a fecal collection and evaluation system;
a urinalysis collection and evaluation system;
a pulse oximetry system;
an ambient condition monitoring system, wherein the ambient condition is a temperature of a surrounding;
a fingerprint scanning system; and
a global positioning system (GPS).
